(19) United States
(12) Patent Application Publication
(10) Pub. No.: US 2020/0345268 A1
(43) Pub. Date: Nov. 5, 2020

(54) ISOTOPIC BIOMARKERS OF ORGANIC ACIDEMIAS

(71) Applicants: Charles P. VENDITTI, Bethesda, MD (US); Irini MANOLI, Bethesda, MD (US); The United States of America, as represented by the Secretary, Dept.of Health and Human Services, ROCKVILLE, MD (US)

(72) Inventors: Charles P. VENDITTI, Potomac, MD (US); Irini MANOLI, Bethesda, MD (US)

(21) Appl. No.: 16/758,009

(22) PCT Filed: Oct. 31, 2018
(86) PCT No.: PCT/US2018/058515

§ 371 Date: Apr. 21, 2020

Related U.S. Application Data
(60) Provisional application No. 62/579,247, filed on Oct. 31, 2017.

(51) Int. Cl. A61B 5/083; G01N 33/497

(52) U.S. Cl. CPC A61B 5/0836; G01N 33/497; A61K 45/06; G01N 2800/7066; G01N 2800/52

(57) ABSTRACT

Methods of using isotopic biomarkers in determining the efficacy of a treatment for an organic acidemia in a subject are disclosed herein. Methods of using isotopic biomarkers in determining the efficacy of a liver-directed treatment for an organic acidemia in a subject are likewise disclosed herein.

### STATEMENT OF GOVERNMENT INTEREST

This invention was made with Government support under project number 1ZIAHG200318-13 by the National Institutes of Health. The Government has certain rights in the invention.

### FIELD

The present disclosure relates, in general, to methods of using isotopic biomarkers in determining the severity of an organic acidemia and response to therapy, and, more particularly, to methods of using isotopic biomarkers in determining the efficacy of a treatment, for example, a liver-directed treatment for an organic acidemia.

### BACKGROUND

Methylmalonic acidemia (“MMA”) is an autosomal recessive disorder caused by defects in the mitochondrial localized enzyme methylmalonyl-CoA mutase (MUT). The estimated incidence of MMA is 1 in 25,000-48,000. MUT is an enzyme that catalyzes the conversion of L-methylmalonyl-CoA to succinyl-CoA. This reaction is one of several enzymatic reactions required to metabolize branch chain amino acids, odd chain fatty acids, cholesterol, and propionate produced by the gut flora (Chandler, et al. 2005 Mol Genet Metab 86:34-43). MUT deficiency, the most common cause of isolated MMA, is characterized by the accumulation of methylmalonic acid and other disease-related metabolites (Manoli and Venditti, Genereviews). The disease is managed by dietary restriction of amino acid precursors and symptomatic treatment of various multiorgan complications, but it lacks definitive therapy. MMA is associated with metabolic instability, growth failure, intellectual impairment, pancreatitis, strokes, and kidney failure, and it can be lethal, even when patients are being properly managed, underscoring the need for new therapies for this disease.

Current MMA treatments include, but are not limited to, dietary restrictions, liver transplantation, and combined liver and kidney transplantation.

The MUT enzyme requires adenosylcobalamin (Ado-Cbl) as coenzyme. Therefore, the methylmalonic acid metabolism is inevitably linked to vitamin B12 (cobalamin), its adequate intake and correct uptake, transport and intracellular metabolism. The cblA, cblB and the variant 2 form of cblD complementation groups are caused by defects in enzymatic steps involved in Ado-Cbl synthesis. The cblC, cblD, cblF, cblJ complementation groups are associated with defective methyl-cobalamin synthesis, as well, and are associated with combined MMA- and homocystin-uria. Cobalamin C (cblC) is the most common disorder of cobalamin metabolism. (Carrillo et al. GeneReviews 2013, Disorders of Intracellular Cobalamin Metabolism.) CblC typically presents in the neonatal period with neurological deterioration, failure to thrive, cytopenias, and multisystem pathology including renal and hepatic dysfunction. (Weisfeld-Adams et al. Mol Genet Metab. 2010 February; 99(2): 116-123.)

The related disorder, propionic acidemia (“PA”), is an autosomal recessive disorder caused by defects in propionyl CoA carboxylase (“PCC”) of either the propionyl CoA carboxylase alpha (PCCA) or beta subunits (PCCB). PCC is inactive in affected individuals with either PCCA or PCCB deficiency. Patients with PA cannot metabolize branch chain amino acids, odd chain fatty acids, cholesterol, and propionate produced by the gut flora (Schechlechov and Venditti, Genereviews). The condition leads to an abnormal buildup of propionic acid, 2-methylcitric acid, and 3-hydroxypropionic acid that can accumulate to toxic levels in the body. This accumulation damages the brain, nervous system and heart, causing the serious health problems associated with PA. The disease is managed by dietary restriction of amino acid precursors and cofactors, but lacks definitive therapy. PA is associated with metabolic instability, seizures, pancreatitis, strokes, and a propensity to develop hyperammonemia. Like MMA, PA can be lethal, even when patients are being properly managed, underscoring the need for new therapies for this disease.

Current PA treatments include, but are not limited to, dietary restrictions, and elective liver transplantation.

Isotope tracers have been used to probe propionate oxidation and measure in vivo enzymatic activity as a prognostic indicator in disorders of propionate metabolism. (Thompson et al. Eur J Pediatr (1990) 149:408-411). In Thompson et al., propionate isotopomer was administered intravenously, which is invasive and especially difficult for pediatric patients and patients with neurocognitive impairment. Barshop investigated the metabolism of propionate in human subjects using oral bolus administration of 1-¹³C-propionate. (Barshop et al. Pediatr Res. 1991, 30(1):15-22) Barshop et al used a large dose of oral 1-¹³C-propionate of 100 μmol/kg, while CO₂ production was estimated, not measured, based on resting energy expenditure (REE, kcal/hr), which was in turn estimated using the Bateman formula, coefficients derived from age- and sex-dependent basal metabolic rates in normal control populations, and body surface area. The REE was not directly measured using calorimetry/metabolic cart. An estimation, rather than direct measurement of the REE, constitutes a source of error in the ability to calculate oxidation capacity for 1-¹³C-propionate, particularly because in the MMA patient population, the resting energy expenditure and CO₂ production are known to be lower than in healthy, age-matched controls, mainly because of low muscle mass and renal failure (Hauser et al, 2011 Am J Clin Nutr. 93(1):47-5). Further, the REE can also vary significantly in this fragile patient population, depending on their overall health status and intercurrent illnesses (Feillet et al, J Pediatr. 2000 May; 136(5):659-63; Bodamer et al, Eur J Pediatr. 1997 August; 156 Suppl 1:S24-8). Moreover, some MMA patients suffer from a debilitating movement disorder caused by a metabolic stroke of the globus pallidi in the basal ganglia, a rare but severe complication of the disease, which further complicates predictions of REE.

There is a need for better therapies, as well as better methods for monitoring and/or determining the efficacy of therapies, for MMA, PA, cobalamin metabolic disorders and other organic acidemias in a subject. Biochemical measures have intrinsic limitation as outcome parameters, because plasma/serum MMA, 2-methylcitric, and propionylcarnitine are affected by dietary intake, renal function and carnitine supplementation, lead to high variability and inconsistent response to interventions, such as liver or combined liver/kidney transplantation.

### BRIEF SUMMARY

Methods of monitoring and/or determining efficacy of a treatment for an organic acidemia in a subject are provided. In one aspect, the organic acidemia is MMA or PA. In another aspect, the treatment is liver-directed treatment, such as gene or mRNA therapy. In yet another embodiment, the treatment is systemic AAV gene therapy, mRNA therapy, enzyme replacement therapy, nuclease free AAV based genome editing designed to introduce the MUT gene into the albumin locus or other locations, or conventional CAS/CRISPR approaches to restore or activate MUT activity. Methods of determining the effects of hepatic mitochondrial dysfunction in patients suffering from an organic acidemia are provided. Further, methods are provided for monitoring therapeutic interventions for other metabolic disorders, comprising administration of isotopomers.

In one aspect, the invention discloses that the degree of metabolism is reflected in isotope breath tests using isotope-labeled metabolites, which correlates with organic acidemia severity. In one embodiment, the isotope-labeled metabolite is 1-¹³C-propionate, 1-¹³C-glycine, or 1-¹³C-methionine. In particular, a method for monitoring and/or determining the efficacy of a treatment for an organic acidemia in a subject is disclosed. The method comprises the steps of, prior to, and after a treatment, administering to the subject a composition having isotope-labeled propionate, collecting breath samples from the subject at a plurality of time points, measuring ¹³CO₂/¹²CO₂ ratio of the breath samples, and determining propionate oxidation rate prior to an intervention or treatment and/or after the treatment. An increase in the propionate oxidation rate after the treatment indicates efficacy of the treatment. The propionate oxidation rate is determined based on the measured ¹³CO₂/¹²CO₂ ratio and the measured CO₂ production rate of the subject. The composition having isotope-labeled propionate may, in certain embodiments, be administered by oral or gastric route. In one embodiment, the treatment is a liver-directed treatment. In another embodiment, the treatment comprises administering to the subject a liver-directed gene transfer vector of a conventional or integrating vector or genome editing reagents designed to correct or activate MUT expression. In another embodiment, the treatment comprises administering to the subject a liver-directed mRNA therapy. In yet another embodiment, the treatment comprises administering to the subject systemic gene or mRNA therapy or enzyme replacement therapy.

In another aspect, the present disclosure provides methods for real time monitoring of the degree of metabolism reflected in isotope breath tests using isotope-labeled metabolites. Disclosed real time methods for monitoring and/or determining the efficacy of a treatment provide tremendous practical advantages for care providers because the efficacy of treatment can be tested non-invasively, at the patient's bed-side, with results provided in 2 hours or less.

In another aspect, the isotope-labeled propionate oxidation rate after a treatment is compared with a predetermined rate, wherein an increase in the isotope-labeled propionate oxidation rate after the treatment compared to the predetermined rate indicates efficacy of the treatment. In this embodiment, administration of isotope-labeled propionate before the treatment might not be necessary, if increased activity is noted. For example, in the case of a patient with a severe genetic form of MMA or PA, who has received a liver transplant prior to testing.

In another aspect, the invention provides a method for improving hepatic enzyme activity in a subject having an organic acidemia. An increase in the isotope-labeled propionate oxidation rate after the treatment indicates efficacy for improving compromised hepatic enzyme activity associated with the organic acidemia.

In another aspect, the invention provides a method for diagnosing hepatic mitochondrial dysfunction in a subject suffering from an organic acidemia. A decrease in the isotope-labeled glycine or methionine oxidation rate compared to a predetermined rate indicates that the subject is suffering from hepatic mitochondrial dysfunction.

In another aspect, non-invasive methods that combine direct measurement of the REE with recovery of CO₂ after label administration are provided to accurately probe the response of MMA and PA patients to 1-¹³C-propionate.

In another aspect, the invention provides a kit for treating or diagnosing an organic acidemia. The kit comprises a predetermined amount of isotope-labeled propionate, a plurality of breath collection bags, guidance for testing before, during, and/or after treatment, and guidance for interpreting the test results.

### BRIEF DESCRIPTION OF THE DRAWINGS

FIG. 1 (A) Overview of the strategy used to generate Mut^{-/-}; Tg^{INS-MCK-Mut} mice (B) survival compared to Mut^{-/-} mice (C) weight gain on varied diets (D) the phenotypic appearance of Mut^{-/-}; Tg^{INS-MCK-Mut} mice compared to control littermates fed a regular or high-fat diet (E) and (F) Mut mRNA expression in the various tissues from Mut^{-/-}; Tg^{INS-MCK-Mut} mice (G) plasma methylmalonic acid in the Mut^{-/-}; Tg^{INS-MCK-Mut} mice and the effect of diet (H) 1-¹³C-propionate oxidation in the Mut^{-/-}; Tg^{INS-MCK-Mut} mice (I) 1-¹³C-methionine oxidation in the Mut^{-/-}; Tg^{INS-MCK-Mut} mice (J) 1-¹³C-glycine oxidation in the Mut^{-/-}; Tg^{INS-MCK-Mut} mice.

FIG. 2 shows hepatic ultrastructural changes in Mut^{-/-}; Tg^{INS-MCK-Mut} mice (A,B) compared to a Mut^{+/-}; Tg^{INS-MCK-Mut} control (C).

FIG. 3 shows diminished hepatic electron transport chain immunoreactive enzyme (A) and activity (B) in Mut^{-/-}; Tg^{INS-MCK-Mut} mice compared to a Mut^{+/-}; Tg^{INS-MCK-Mut} control.

FIG. 4 shows renal tubular histological (A) and ultrastructural changes in Mut^{-/-}; Tg^{INS-MCK-Mut} mice (B) compared to a Mut^{+/-}; Tg^{INS-MCK-Mut} control (C). Impaired filtration (D) and increased plasma lipocalin 2 (E) accompany the renal disease.

FIG. 5 shows improved (A) growth, (B) reduction in serum methylmalonic acid concentrations and (C) increased recovery of 1-¹³C-propionate after treatment of Mut^{+/-}; Tg^{INS-MCK-Mut} mice with a MUT AAV9 gene therapy vector.

FIG. 6 shows 1-¹³C-propionate recovery rate in different MMA subtypes where CblA and Mut⁻ are milder forms of MMA and typically responsive to vitamin B12. Mut° MMA patients, in contrast, are more severe clinically and biochemically. As a group, the Mut° MMA patients have impaired 1-¹³C-propionate oxidation compared to controls and other forms of MMA such as CblA and Mut⁻.

FIG. 7 shows effects of organ transplantation on 1-¹³C-propionate recovery rate. Mut_LKT indicates MMA patients that have received a combined liver-kidney transplant, whereas Mut_KT indicates MMA patients that received only a kidney transplant. Mut° indicates MMA patients who have not been transplanted. As can be appreciated, the Mut_LKT but not Mut_KT patients have restored ability to oxidize 1-¹³C-propionate, showing that the liver, in humans, is the main organ responsible for propionate metabolism.

FIG. 8 shows 1-¹³C-propionate recovery rate pre- (Pre_LKT) and post—liver/kidney transplant (Post_LKT) in a patient with mut° MMA. Note that Post_LKT, 1-¹³C-propionate oxidation is restored.

FIG. 9 shows method reproducibility. Hv1 and Hv2 indicate the same heathy volunteer control who was studied on two different occasions over a one year period. The third and fourth lines, open versus filled diamonds, represent a Mut_LKT patient who was studied when the plasma level methylmalonic acid level was either 1741 or 2246 umol/l with very similar results. The fifth line, filled square, is a Mut MMA patient with a partial liver transplant and kidney transplant (Mut_pLKT) who was studied on two different occasions over a two years period with varying levels of methylmalonic acid in her plasma (719 vs 2260 umol/L) yet demonstrated nearly identical 1-¹³C-propionate oxidation, which is why the line appears to contain only one symbol as the values for each timepoint between the two studies were nearly identical. The other lines represent patients as indicated. The aggregate coefficient of variation for all the studies compared to repeat studies was 2.28-3.34%.

FIG. 10 shows the variability of serum methymalonic acid levels in a cohort of MMA patients.

FIG. 11 shows 1-¹³C-propionate oxidation rate in PA patients. 1-¹³C-propionate oxidation in PA patients correlates with biochemical severity. The squares show the values from a mild patient whereas the other lines are from those more severely affected.

FIG. 12A shows, for MMA patient #1, real time BREATHID metabolic monitoring compared to simultaneous metabolic monitoring with bag collected exhalation measured by Isotope Ratio Mass Spectroscopy.

FIG. 12B shows, for MMA patient #2, real time BREATHID metabolic monitoring compared to simultaneous metabolic monitoring with bag collected exhalation measured by Isotope Ratio Mass Spectroscopy.

FIG. 12C shows, for MMA patient #3, real time BREATHID metabolic monitoring compared to simultaneous metabolic monitoring with bag collected exhalation measured by Isotope Ratio Mass Spectroscopy.

FIG. 12D shows, for MMA patient #4, real time BREATHID metabolic monitoring compared to simultaneous metabolic monitoring with bag collected exhalation measured by Isotope Ratio Mass Spectroscopy.

FIG. 12E shows, for MMA patient #5, real time BREATHID metabolic monitoring compared to simultaneous metabolic monitoring with bag collected exhalation measured by Isotope Ratio Mass Spectroscopy.

FIG. 12F shows, for MMA patient #6, real time BREATHID metabolic monitoring compared to simultaneous metabolic monitoring with bag collected exhalation measured by Isotope Ratio Mass Spectroscopy.

FIG. 13A shows, for PA patient #1, real time BREATHID metabolic monitoring compared to simultaneous metabolic monitoring with bag collected exhalation measured by Isotope Ratio Mass Spectroscopy.

FIG. 13B shows, for PA patient #2, real time BREATHID metabolic monitoring compared to simultaneous metabolic monitoring with bag collected exhalation measured by Isotope Ratio Mass Spectroscopy.

FIG. 13C shows, for PA patient #3, real time BREATHID metabolic monitoring compared to simultaneous metabolic monitoring with bag collected exhalation measured by Isotope Ratio Mass Spectroscopy.

FIG. 13D shows, for PA patient #4, real time BREATHID metabolic monitoring compared to simultaneous metabolic monitoring with bag collected exhalation measured by Isotope Ratio Mass Spectroscopy.

FIG. 13E shows, for PA patient #5, real time BREATHID metabolic monitoring compared to simultaneous metabolic monitoring with bag collected exhalation measured by Isotope Ratio Mass Spectroscopy.

FIG. 14 shows metabolic pathways affected in PA and MMA and the egress of ¹³CO₂ from administered 1-¹³C-propionate.

FIG. 15 shows positioning of the IDcircuit™.

FIG. 16A shows the Delta Over Baseline difference between the Delta value (based on a ratio of ¹³CO₂/¹²CO₂) in the test specimen and the corresponding baseline sample for MMA patient #2 as measured with bag collected exhalation measured by Isotope Ratio Mass Spectroscopy.

FIG. 16B shows the Delta Over Baseline difference for the same patient as in FIG. 16A as measured with BREATHID.

FIG. 16C shows the cumulative percent of dose metabolized for the same MMA patient as in FIG. 16A as measured with bag collected exhalation measured by Isotope Ratio Mass Spectroscopy.

FIG. 16D shows the cumulative percent of dose metabolized for the same MMA patient as in FIG. 16A as measured with BREATHID.

FIG. 17A shows the Delta Over Baseline for PA patient #5 as measured with bag collected exhalation measured by Isotope Ratio Mass Spectroscopy.

FIG. 17B shows the Delta Over Baseline difference for the same patient as in FIG. 17A as measured with BREATHID.

FIG. 17C shows the cumulative percent of dose metabolized for the same PA patient as in FIG. 17A as measured with bag collected exhalation measured by Isotope Ratio Mass Spectroscopy.

FIG. 17D shows the cumulative percent of dose metabolized for the same MMA patient as in FIG. 17A as measured with BREATHID.

FIGS. 18A-B show 1-¹³C pyruvate oxidation rate in MMA mutant mice and sex matched littermate controls.

FIGS. 19A-B show 1-¹³C leucine oxidation rate in MMA mutant mice and sex matched littermate controls.

FIGS. 20A-B show 1-¹³C octanoate oxidation rate in MMA mutant mice and sex matched littermate controls.

FIGS. 21A-B show 1-¹³C palmitate oxidation rate in MMA mutant mice and sex matched littermate controls.

FIGS. 22A-B show 1-¹³C phenylalanine oxidation rate in wild type mice.

### DETAILED DESCRIPTION OF CERTAIN EMBODIMENTS

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

### Definitions

Unless defined otherwise, technical and scientific terms used herein have the same meaning as commonly understood by one of ordinary skill in the art to which this invention belongs. Any methods, devices, and materials similar or equivalent to those described herein can be used in the practice or testing of the invention.

As used in this application, including the appended claims, the singular forms “a,” “an,” and “the” include plural references, unless the content clearly dictates otherwise, and are used interchangeably with “at least one” and “one or more.”

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

The term “subject” or “patient”, as used herein, refers to a domesticated animal, a farm animal, a primate, or a mammal, for example, a human.

The terms “determining”, “determination”, “detecting”, or the like are used interchangeably herein and refer to the detecting or quantitation (measurement) of a molecule using any suitable method.

As used herein, the terms “treat,” “treating”, and “treatment” mean to alleviate symptoms, eliminate the causation of symptoms either on a temporary or permanent basis, or to prevent or slow the appearance of symptoms of the named disorder or condition. Treatment is, in certain embodiments, directed at a subject or patient suffering from an organic acidemia, and may reduce the severity of the organic acidemia, or retard or slow the progression of the organic acidemia. Standard treatments include, but are not limited to, a limited protein/high carbohydrate diet, intravenous fluids, amino acid substitution, vitamin supplementation, carnitine, induced anabolism, and tube-feeding. Exemplary treatments include more aggressive treatments like liver transplant, combined liver and kidney transplant, and emerging therapies involving gene, mRNA, cell, small molecules, read-through agents, stem cell therapies, genome editing, chaperones, ERT, microbiome, or any other processes that could improve MUT or PCC activity or propionate oxidation or associated mitochondrial dysfunction. In one embodiment, the treatment is liver-directed treatment.

As used herein, the term “organic acidemia” refers to a group of inheritable metabolic disorders which disrupt normal amino acid metabolism, particularly branched-chain amino acids, causing a buildup of acids which are usually not present. Exemplary organic acidemias include, but not limited to, methylmalonic acidemia (MMA), propionic acidemia (PA), isovaleric acidemia (IVA), glutaric aciduria type 1 (GA1), beta-ketothiolase deficiency (BKT), 3-methylcrotonyl-CoA carboxylase deficiency (3-MCC), 3-hydroxy-3-methylglutaryl-CoA lyase deficiency (HMG), 3-Methylglutaconic acidemia or 3-Methylglutaconyl-CoA Hydratase Deficiency (MGA), D-2 Hydroxyglutaric Aciduria (D2-HGA), Isobutyryl-CoA Dehydrogenase Deficiency 3-Hydroxyisobutyric aciduria (ICBD), L-2-Hydroxy-glutaricaciduria (L2HGA), Malonyl-CoA Decarboxylase Deficiency aka Malonic Acidemia (MA), Multiple carboxylase deficiency (MCD, holocarboxylase synthetase), and 3-Hydroxyisobutyryl-CoA Hydrolase Deficiency (HIBCH).

MMA is an autosomal recessive disorder most commonly caused by reduced or absent activity of the mitochondrial localized enzyme, methylmalonyl-CoA mutase (MUT), and results in the accumulation of methylmalonic acid. Defects in the transport and metabolism of the cofactor for MUT, 5-deoxyadenosylcobalamin, also can cause MUT deficiency. These disorders include cblA, cblB and cblD class of MMA, and mutations in the corresponding genes, MMAA (cblA), MMAB (cblB), and MMADHC (cblD). In addition, MMACHC (cblC), LMBRD1 (cblF), ABCD4 (cblJ), TC2 (transcobalamin 2), CD320, AMN (encoding amnionless), TCBLR(transcobalamin receptor) or Imerslund-Graesbeck forms of combined MMAemia-hyperhomocysteinemia may also cause reduced MUT enzyme activity because of B12 deficiency.

PA is an autosomal recessive disorder caused by defects in propionyl-CoA carboxylase (PCC) of either the propionyl CoA carboxylase alpha (PCCA) or beta subunits (PCCB) and resulting in the accumulation of propionic acid and related metabolites.

As used herein, the term “cobalamin metabolic and transport disorder” refers to disorders associated with cobalamin deficiency. Exemplary cobalamin metabolism disorders include, but not limited to, MMACHC (cblC), MMADHC(cblD), LMBRD1(cblF), ABCD4(cblJ), TC2, CD320, AMN, TCBLR (transcobalamin receptor) or Imerslund-Gräesbeck forms of combined MMAemia-hyperhomocysteinemia Diagnosis of disorders of intracellular cobalamin metabolism with increased methymalonic acid is confirmed by identification of biallelic pathogenic variants in one of the following genes (associated complementation groups indicated in parentheses): MMACHC (cblC), MMADHC (cblD and cblD variant 1), LMBRD1 (cblF), and ABCD4 (cblJ). CblC is the most common cobalamin metabolic disorder.

As used herein, the term “disorder of propionate metabolism” refers to disorders associated with the chemical reactions and pathways involving propionate. Exemplary disorders of propionate metabolism include, but are not limited to, MMA and PA.

As used herein, the term “efficacy” refers to any increase in the therapeutic benefit to the subject.

As used herein, the term “biomarker” refers to a measurable parameter, or combination of parameters, that can be used as an indicator of a biological state.

As used herein, the term “propionate” encompasses salts and esters of propionic acid or derivative thereof, such as sodium propionate. Thus, propionate can be administered as sodium propionate or in any of its other forms, e.g. salts and esters thereof, as well as combination thereof.

As used herein, the term “predetermined level”, “predetermined standard level”, “reference standard level”, or “reference level” refers to an accepted level of the biomarker used to compare the biomarker level derived from a sample of a subject. In one embodiment, the predetermined standard level of the biomarker indicates an unaffected, i.e., non-disease, state of a subject who does not have an organic acidemia.

As used herein, the term “metabolite” refers to the reactants (e.g., precursors), intermediates, and products of metabolic transformations.

As used herein, the term “decrease” refers to a level of the biomarker smaller in value. As used herein, the term “increase” refers to a level of the biomarker larger in value. A decrease of propionate oxidation rate refers to a level of the propionate oxidation rate smaller in value. An increase of propionate oxidation rate refers to a level of the propionate oxidation rate larger in value.

### Methods

The invention advantageously provides a non-invasive isotope breath test for monitoring, for example, mitochondrial dysfunction in MMA and PA. In one embodiment, the invention replaces known invasive monitoring procedures, such as muscle, liver, or renal biopsies. In another embodiment, the invention provides a more sensitive test for clinical treatment responses, i.e., can monitor responses to therapeutics before showing clinical symptoms, for example, of hepatic mitochondrial function.

In another aspect, the present disclosure provides methods for real time monitoring of the degree of metabolism reflected in isotope breath tests using isotope-labeled metabolites. Disclosed real time methods for monitoring and/or determining the efficacy of a treatment provide tremendous practical advantages for care providers because the efficacy of treatment can be tested non-invasively, at the patient's bed-side, with results provided in 2 hours or less.

By enabling care providers to non-invasively measure the efficacy of treatment in real time within hours after treatment, care providers are able to quickly determine whether the administered treatment has taken effect or if further intervention is necessary.

In certain embodiments, the invention provides an isotopic breath test to measure the effects of any intervention on hepatic MUT or PCC activity and the effects of hepatic MUT or PCC deficiency, and the secondary hepatic mitochondriopathy associated with MUT and PCC deficiency. In some embodiments, the invention provides an isotopic breath test that could be used to monitor the effects of gene, mRNA, cell, small molecule, microbiome, or any other process that could improve MUT or PCC activity or propionate oxidation or associated mitochondrial dysfunction. Such monitoring would be helpful for vitamin B12 deficiency and any enzymes that depend on vitamin B12 and possibly biotin. The isotopic breath test can be similarly used to monitor treatment(s) for methylmalonic and propionic acidemias.

In certain embodiments, the invention could be applied to propionate oxidation disorders, including all forms of propionic acidemia, methylmalonic acidemia, cobalamin defects (cblA, B, C, D, F, J; TC2, TCBLR, AMN), vitamin B12 and biotin deficiency; disorders that affect hepatic mitochondrial metabolism; to test for effects of drugs that affect hepatic metabolism such as HIV medicines, statins, metformin, and any therapies directed toward these disorders, including but not limited to, hepatic gene therapy with any vector (AAV, adenovirus, lentivirus), cell therapy, enzyme-specific chaperonins, engineered microbes/microbiome, mRNA therapy, enzyme replacement therapy, genome editing using conventional or nuclease free AAV approaches, small molecules, read-through agents, stem cell therapies, or any other processes that could improve MUT or PCC activity or propionate oxidation or associated mitochondrial dysfunction. In one embodiment, the method is applied for any form of MMA or PA.

In yet another embodiment, the metabolism of 1-¹³C isotopomers for monitoring of therapeutic interventions for other related metabolic disorders is provided. Many metabolic disorders where hepatic metabolism of the tracer into CO₂, representing substrate oxidation, are candidates for non-invasive isotopic monitoring to ascertain efficacy of therapeutic intervention which might include liver directed gene therapy using AAV vectors, enzyme replacement therapy, genome editing, mRNA therapy, microbiome manipulations, chaperones, small molecule activators, and cofactors. Table 2 lists examples of the disorders, labels, and dosing.

In some embodiments, comparing the isotope-labeled propionate oxidation rate in a subject to a predetermined or reference propionate oxidation rate comprises generating a cumulative percentage dose recovery (CPDR) curve for the subject and comparing at least one parameter of said CPDR curve to at least one parameter of a predetermined or reference CPDR curve. Such curves depict the amount of the labeled substrate that was metabolized in % dose (cumulative percentage of the administered dose recovered over time), as measured in a breath. The cumulative recovery of labeled CO₂ in a breath can be calculated as the area under the curve (AUC) of PDR. In some embodiments, the parameter is one or more CPDR values at selected time points, for example, CPDR values at 30, 40 and/or 45 minutes. In some embodiments, the parameter is one or more CPDR values at selected time points from the time administering the isotope-labeled propionate to the subject. In some embodiments, the parameter is the peak height.

In other embodiments, comparing isotope-labeled propionate oxidation rate in the subject prior to and after a treatment comprises generating a cumulative percentage dose recovery (CPDR) curve for prior to and after the treatment, respectively, and comparing at least one parameter of CPDR curve prior to the treatment to at least one parameter of CPDR curve after the treatment. Such curves depict the amount of the labeled substrate that was metabolized in % dose (cumulative percentage of the administered dose recovered over time), as measured in a breath. The cumulative recovery of labeled CO₂ in a breath can be calculated as the area under the curve (AUC) of PDR. In some embodiments, the parameter is one or more CPDR values at selected time points, for example, CPDR values at 30, 40 and/or 45 minutes. In some embodiments, the parameter is one or more CPDR values at selected time points from the time administering the isotope-labeled propionate to the subject. “After a treatment” may include after a stage or step of a treatment.

In some embodiments, comparing isotope-labeled propionate oxidation metabolism in the subject to a predetermined or reference propionate oxidation rate comprises generating a delta over baseline (DOB) curve and comparing at least one parameter of said DOB curve to at least one parameter of a predetermined reference DOB curve. Such curves depict the difference between the isotope ratio (for example, ¹³CO₂/¹²CO₂) in a test sample collected at a certain time point and the corresponding ratio in a baseline sample. In some embodiments, the parameter is one or more DOB values at selected time points. In some embodiments, the parameter is one or more DOB values at selected time points from the time administering the isotope-labeled propionate to the subject. In some embodiments, the parameter is the peak height.

In some embodiments, comparing isotope-labeled propionate oxidation metabolism in the subject prior to and after a treatment comprises generating a delta over baseline (DOB) curve prior to and after the treatment, respectively, and comparing at least one parameter of the DOB curve prior to the treatment to at least one parameter of the DOB curve after the treatment. Such curves depict the difference between the isotope ratio (for example, ¹³CO₂/¹²CO₂) in a test sample collected at a certain time point prior to and after the treatment. In some embodiments, the parameter is one or more DOB values at selected time points. In some embodiments, the parameter is one or more DOB values at selected time points from the time administering the isotope-labeled propionate to the subject. In one embodiment, the parameter is the maximal DOB value. In one embodiment is the time at which DOB is maximal.

PDR curves represent normalization of the DOB per subject taking into consideration the subject's CO₂ production rate. The subject's CO₂ production rate may be estimated based on height and weight of the subject and the amount of substrate administered. In one embodiment, the subject's CO₂ production is measured on the same day prior to administering the isotope-labeled metabolite, such as sodium 1-¹³C-propionate.

In some embodiments, the invention provides a method for determining the efficacy of a treatment for an organic acidemia in a subject. The method comprises the steps of prior to the treatment: (i) by oral or gastric route, administering to the subject a composition having isotope-labeled propionate in an amount of about 1 μmol/kg to about 100 μmol/kg body weight; (ii) collecting breath samples from the subject at a plurality of time points after step (i); (iii) measuring the ¹³CO₂/¹²CO₂ ratio of the breath samples from step (ii); (iv) determining a first isotope-labeled propionate oxidation rate based on the measured ¹³CO₂/¹²CO₂ ratio of step (iii) and measured CO₂ production rate. In one embodiment, the CO₂ production rate is measured by an indirect calorimetry cart on the same day prior to step (i). The method further comprises the steps of following the treatment: (v) by oral or gastric route, administering to the subject a composition having isotope-labeled propionate in the amount of about 1 μmol/kg to about 100 μmol/kg body weight; (vi) collecting breath samples from the subject at a plurality of time points after the step (v); (vii) measuring ¹³CO₂/¹²CO₂ ratio of the breath samples from step (vi); (viii) determining a second isotope-labeled propionate oxidation rate based on the measured ¹³CO₂/¹²CO₂ ratio of step (vii) and measured CO₂ production rate. In one embodiment, the CO₂ production rate is measured by an indirect calorimetry cart on the same day prior to step (v). The method further comprise the step of comparing the first isotope-labeled propionate oxidation rate with the second isotope-labeled propionate oxidation rate, wherein an increase in the second isotope-labeled propionate oxidation rate compared to the first isotope-labeled propionate oxidation rate indicates efficacy of the treatment. In some embodiments, the isotope-labeled propionate is administered in the amount of about 0.5 mg/kg or 5.15 μmol/kg body weight. In some embodiments, the composition having isotope-labeled propionate is administered via a single drink. In yet another embodiment, the composition having isotope-labeled propionate is administered more than one drink over time.

In one embodiment, the treatment is a liver-directed treatment. In another embodiment, the treatment comprises administering to the subject a liver-directed gene transfer vector. In another embodiment, the treatment is liver transplantation or combined liver and kidney transplantation. In another embodiment, the treatment is selected from the group consisting of gene therapy, cell therapy, small molecules, enzyme specific chaperonins, engineered microbes/microbiome, mRNA therapy, enzyme replacement therapy, and genome editing therapies. In another embodiment, the treatment is selected from the group consisting of genome editing using conventional or nuclease-free AAV approaches, read-through agents, stem cell therapies, chaperones, ERT, or any other processes that could improve MUT or PCC activity or propionate oxidation or associated mitochondrial dysfunction.

In another embodiment, the organic acidemia is selected from the group consisting of methylmalonic acidemia (MMA), propionic acidemia (PA), isovaleric acidemia, glutaric aciduria type 1 (GA1), beta-ketothiolase deficiency (BKT), 3-methylcrotonyl-CoA carboxylase deficiency (3-MCC), 3-hydroxy-3-methylglutaryl-CoA lyase deficiency (HMG), 3-Methylglutaconic acidemia or 3-Methylglutaconyl-CoA Hydratase Deficiency (MGA), D-2 Hydroxyglutaric Aciduria (D2-HGA), Isobutyryl-CoA Dehydrogenase Deficiency 3-Hydroxyisobutyric aciduria (ICBD), L-2-Hydroxy-glutaricaciduria (L2HGA), Malonyl-CoA Decarboxylase Deficiency aka Malonic Acidemia (MA), Multiple carboxylase deficiency (MCD, holocarboxylase synthetase), and 3-Hydroxyisobutyryl-CoA Hydrolase Deficiency (HIBCH). In another embodiment, the organic acidemia is methylmalonic acidemia or propionic acidemia.

In another embodiment, the organic acidemia is a disorder of propionate metabolism or a cobalamin metabolic and transport disorder causing MUT deficiency. In another embodiment, the disorder of propionate metabolism is caused by isolated methylmalonyl-CoA mutase (MUT), MMAA, MMAB, or MMADHC deficiency; or mut, cblA, cblB, cblD variant 2 classes of MMA. In another embodiment, the cobalamin metabolic and transport disorders is selected from the group consisting of MMACHC, MMADHC, LMBRD1, ABCD4, TC2, CD320, AMN deficiency, TCBLR and Imerslund-Graesbeck forms of combined MMAemia-hyperhomocysteinemia.

In one embodiment, the isotope-labeled propionate is administered in the amount of equal to or less than about 100 μmol/kg body weight, equal to or less than about 50 μmol/kg body weight, equal to or less than about 40 μmol/kg body weight, equal to or less than about 30 μmol/kg body weight, equal to or less than about 20 μmol/kg body weight, equal to or less than about 10 μmol/kg body weight, equal to or less than about 9 μmol/kg body weight, equal to or less than about 8 μmol/kg body weight, equal to or less than about 7 μmol/kg body weight, equal to or less than about 6 μmol/kg body weight, equal to or less than about 5 μmol/kg body weight, equal to or less than about 4 μmol/kg body weight, equal to or less than about 3 μmol/kg body weight, equal to or less than about 2 μmol/kg body weight, equal to or less than about 1 μmol/kg body weight, 0.1-9 μmol/kg body weight, 0.1-8 μmol/kg body weight, 0.1-7 μmol/kg body weight, 0.1-6 μmol/kg body weight, 0.1-5 μmol/kg body weight, 0.1-4 μmol/kg body weight, 0.1-3 μmol/kg body weight, 0.1-2 μmol/kg body weight, or 0.1-1 μmol/kg body weight. In one embodiment, isotope-labeled propionate is administered in the amount of about 5 μmol/kg body weight. In one embodiment, the isotope-labeled propionate is sodium 1-¹³C-propionate.

In another embodiment, the isotope-labeled propionate is administered in the amount of equal to or less than about 10 mg/kg body weight, equal to or less than about 9 mg/kg body weight, equal to or less than about 8 mg/kg body weight, equal to or less than about 7 mg/kg body weight, equal to or less than about 6 mg/kg body weight, equal to or less than about 5 mg/kg body weight, equal to or less than about 4 mg/kg body weight, equal to or less than about 3 mg/kg body weight, equal to or less than about 2 mg/kg body weight, equal to or less than about 1.0 mg/kg body weight, equal to or less than about 0.9 mg/kg body weight, equal to or less than about 0.8 mg/kg body weight, equal to or less than about 0.7 mg/kg body weight, equal to or less than about 0.6 mg/kg body weight, equal to or less than about 0.5 mg/kg body weight, equal to or less than about 0.4 mg/kg body weight, equal to or less than about 0.3 mg/kg body weight, equal to or less than about 0.2 mg/kg body weight, equal to or less than about 0.1 mg/kg body weight, 0.01-1.0 mg/kg body weight, 0.01-0.9 mg/kg body weight, 0.01-0.8 mg/kg body weight, 0.01-0.7 mg/kg body weight, 0.01-0.6 mg/kg body weight, 0.01-0.5 mg/kg body weight, 0.01-0.4 mg/kg body weight, 0.01-0.3 mg/kg body weight, 0.01-0.2 mg/kg body weight, 0.01-0.1 mg/kg body weight. In one embodiment, isotope-labeled propionate is administered in the amount of about 0.5 mg/kg body weight. In one embodiment, the isotope-labeled propionate is sodium 1-¹³C-propionate.

In one embodiment, the breath samples are collected in collection containers. The collection containers may be in the form of gas-tight bags, which are initially flat at the beginning of the test, and each of which is sequentially filled by the inflow of the breath sample directed to that bag. The collection container may contain one way valve mouthpiece. The mouthpiece may be inserted into the bottom of the collection bag. The mouthpiece may facilitate inflation and establish airtight connections between airway and collection bags in order to reduce room air cross-contamination. The collection container may be made from foil, plastics, and/or glass. In some embodiments, the breath samples may be collected with a commercially available breath sampler. These include, but are not limited to a Quintron™ EasySampler™ (Milwaukee, Wis.). These samplers have a mouthpiece and a collection bag with a one-way valve. The breath samples are trapped in a collection bag or other suitable breath collection device and the contents are injected into an evacuated tube. The use of nasal prongs or a mask to collect expired breaths is also provided.

The breath sample may be taken to a gas analyzer system for analysis using Gas Isotope Ratio Mass Spectrometry or Infrared Spectroscopy to measure the C¹³O₂ content and ratio of C¹³O₂ to endogenous ¹²CO₂.

The breath samples may be collected at time points at selected intervals for up to six hours after administration of a composition having isotope-labeled propionate. In some embodiments, the step of collecting breath samples comprises collecting breath samples at a plurality different time points include at least a first time point and a second time point. In other embodiments, the plurality of time points include at least a first time point, a second time point, and a third time point. In other embodiments, the plurality of time points include at least a first time point, a second time point, a third time point and a fourth time point. In other embodiments, the plurality of time points include at least a first time point, a second time point, a third time point, a fourth time point and a fifth time point. In yet other embodiments, the plurality of time points include at least a first time point, a second time point, a third time point, a fourth time point, a fifth time point and a sixth time point. The time points can be spaced at any desired interval, such as a 15 minute interval, a 20 minute interval or a 30 minute interval. In certain embodiments, the first time point is a 2 minute time point, the second time point is a 3 minute time point, the third time point is a 5 minute time point, the fourth time point is a 10 minute time point, the fifth time point is a 20 minute time point and the sixth time point is a 30 minute time point. In other cases, the first time point is a 5 minute time point, the second time point is a 10 minute time point, the third time point is a 20 minute time point, the fourth time point is a 30 minute time point, the fifth time point is a 45 minute time point and the sixth time point is a 60 minute time point. In some embodiments, the breath samples are collected every 1 to 30 minutes for a one- to four-hour period after administration of a composition having isotope-labeled propionate. In some embodiments, the breath samples are collected every 2 to 15 minutes for one- to two-hour period. Any desired number of time points can be used and the time points can be spaced by any desired time interval.

In some embodiments, the breath test system includes a breath analysis chamber, a breath inlet conduit for conveying exhaled gas from a patient to the breath analysis chamber, and a gas analyzer operative to measure the ratio of ¹³C/¹²C of gas exhaled by the patient. In some embodiments, monitoring an isotope-labeled metabolic product of propionate is performed by continuous measurement. In some embodiments, on-line monitoring is performed, in real time, while a subject is continuing to provide breath for subsequent analyses. U.S. Pat. No. 8,293,187 provides devices and methods for direct measurement of isotopes of expired gases.

In another embodiment, the invention provides a method for determining the efficacy of a treatment for an organic acidemia in a subject. The method comprises the steps of following the treatment: (i) by oral or gastric route, administering to the subject a composition having isotope-labeled propionate in an amount of about 1-100 μmol/kg body weight; (ii) collecting breath samples from the subject at a plurality of time points after step (i); (iii) measuring the ¹³CO₂/¹²CO₂ ratio of the breath samples from step (ii); (iv) determining a first isotope-labeled propionate oxidation rate based on the measured ¹³CO₂/¹²CO₂ ratio of step (iii) and measured CO₂ production rate. In one embodiment, the CO₂ production rate is measured by an indirect calorimetry cart on the same day prior to step (i). The method further comprises comparing the first isotope-labeled propionate oxidation rate with a predetermined rate, wherein an increase in the first isotope-labeled propionate oxidation rate compared to the predetermined rate indicates efficacy of the treatment. In one embodiment, the isotope labeled propionate is sodium 1-¹³C-priopionate.

In another embodiment, the invention provides a method for treating for an organic acidemia in a subject. The method comprises the steps of prior to a treatment: (i) by oral or gastric route, administering to the subject a composition having sodium 1-¹³C-propionate in the amount of about 0.1-about 10.0 mg/kg body weight; (ii) collecting breath samples from the subject at a plurality of time points after the step (i); (iii) measuring ¹³CO₂/¹²CO₂ ratio of the breath samples from step (ii); (iv) determining a first 1-¹³C-propionate oxidation rate based on the measured ¹³CO₂/¹²CO₂ ratio of step (iii) and measured CO₂ production rate. In one embodiment, the CO₂ production rate is measured by an indirect calorimetry cart on the same day prior to step (i). The method further comprises the step of administering a treatment to the subject to improve compromised hepatic enzyme activity associated with the organic acidemia after step (ii). The method further comprises the steps of following the treatment: (v) by oral or gastric route, administering to the subject a composition having sodium 1-¹³C-propionate in the amount of 0.1-10.0 mg/kg body weight; (vi) collecting breath samples from the subject at a plurality of time points after the step (v); (vii) measuring the ¹³CO₂/¹²CO₂ ratio of the breath samples from step (vi); (viii) determining a second 1-¹³C-propionate oxidation rate based on the measured ¹³CO₂/¹²CO₂ ratio of step (vii) and measured CO₂ production rate. In one embodiment, the CO₂ production rate is measured by an indirect calorimetry cart on the same day prior to step (v). The method further comprises the step of discontinuing, altering, or continuing the treatment based on the second 1-¹³C-propionate oxidation rate after treatment compared to the first 1-¹³C-propionate oxidation rate before the treatment. In one embodiment, isotope—labeled propionate in an amount of about 1-10 μg/kg body weight is administered in steps (i) and (v).

In another embodiment, the invention provides a method for measuring hepatic enzyme activity in a subject having an organic acidemia. The method comprises the step of prior to a treatment: (i) by oral or gastric route, administering to the subject a composition having sodium 1-¹³C-propionate in the amount of about 0.1-about 10.0 mg/kg body weight; (ii) collecting breath samples from the subject at a plurality of time points after the step (i); (iii) measuring ¹³CO₂/¹²CO₂ ratio of the breath samples from step (ii); (iv) determining a first 1-¹³C-propionate oxidation rate based on the measured ¹³CO₂/¹²CO₂ ratio of step (iii) and CO₂ production rate measured by an indirect calorimetry cart on the same day prior to step (i). The method further comprises the step of administering a treatment to the subject to improve compromised hepatic enzyme activity associated with the organic acidemia after step (ii). The method further comprises the steps of following the treatment: (v) by oral or gastric route, administering to the subject a composition having sodium 1-¹³C-propionate in the amount of about 0.1-about 10.0 mg/kg body weight; (vi) collecting breath samples from the subject at a plurality of time points after the step (v); (vii) measuring ¹³CO₂/¹²CO₂ ratio of the breath samples from step (vi); (viii) determining a second 1-¹³C-propionate oxidation rate based on the measured ¹³CO₂/¹²CO₂ ratio of step (vii) and CO₂ production rate measured by an indirect calorimetry cart on the same day prior to step (v). The method further comprises the step of discontinuing, altering, or continuing the treatment based on the second 1-¹³C-propionate oxidation rate after treatment compared to the first 1-¹³C-propionate oxidation rate before the treatment. In one embodiment, isotope—labeled propionate in an amount of about 1-10 μg/kg body weight is administered in steps (i) and (v). The method can be applied longitudinally and prospectively.

In one embodiment, the enzyme is selected from the group consisting of methylmalonyl-CoA mutase, propionyl CoA carboxylase, isovaleryl-CoA dehydrogenase, Glutaryl CoA Dehydrogenase, beta-ketothiolase, 3-methylcrotonyl-CoA carboxylase, 3-hydroxy-3-methylglutaryl-CoA lyase, 3-Methylglutaconyl-CoA Hydratase, Isobutyryl-CoA Dehydrogenase, Malonyl-CoA Decarboxylase, Multiple carboxylase, and 3-Hydroxyisobutyryl-CoA Hydrolase.

In one embodiment, the invention provides method for determining efficacy of a treatment for an organic acidemia in a subject. The method comprises the steps of prior to the treatment: (i) by oral or gastric route, administering to the subject a composition having sodium 1-¹³C propionate in the amount of about 0.1-about 10.0 mg/kg body weight; (ii) collecting a first breath sample from the subject with a disposable breath collection kit a first duration after the step (i); (iii) measuring a first ¹³CO₂/¹²CO₂ ratio of the first breath sample. The method further comprises the step of administering the treatment on the subject after step (ii). The method further comprises the steps of following the treatment: (iv) orally administering to the subject a composition having sodium 1-¹³C propionate in the amount of about 0.1-about 10.0 mg/kg body weight; (v) collecting a second breath from the subject sample with a disposable breath collection kit the first duration after the step (iv); (vi) measuring a second ¹³CO₂/¹²CO₂ ratio of the second breath sample. The method further comprises the step of comparing the first ¹³CO₂/¹²CO₂ ratio with the second ¹³CO₂/¹²CO₂ ratio, wherein an increase in the second ¹³CO₂/¹²CO₂ ratio compared to the first ¹³CO₂/¹²CO₂ ratio indicates efficacy of the treatment. In one embodiment, isotope—labeled propionate in an amount of about 0.1-1 mg/kg body weight is administered in steps (i) and (v).

In one embodiment, the invention provides a method for diagnosing hepatic mitochondrial dysfunction in a subject suffering from an organic acidemia. The method comprises the steps of (i) by oral or gastric route, administering to the subject a composition having 1-¹³C methionine or glycine in the amount of about 0.1-about 10.0 mg/kg body weight; (ii) collecting breath samples from the subject at a plurality of time points after the step (i); (iii) measuring ¹³CO₂/¹²CO₂ ratio of the breath samples from step (ii); (iv) determining a first 1-¹³C-propionate oxidation rate based on the measured ¹³CO₂/¹²CO₂ ratio of step (iii) and CO₂ production rate measured by an indirect calorimetry cart on the same day prior to step (i); wherein a decrease in 1-¹³C-propionate oxidation rate compared to a predetermined standard level indicates that the subject is suffering from hepatic mitochondrial dysfunction. In one embodiment, isotope—labeled metabolite in an amount of about 0.1-1 mg/kg body weight is administered in steps (i).

In one embodiment, the invention provides a method for determining the efficacy of a treatment for an organic acidemia in a subject. The method comprises the steps of after the treatment: (i) administering an isotope-labeled metabolite to the subject wherein the isotope-labeled metabolite is 1-¹³C-propionate, 1-¹³C-glycine, or 1-¹³C-methionine; (ii) measuring a level of an isotope-labeled product of the isotope-labeled metabolite in exhaled breath of the subject following administration of the isotope-labeled metabolite; (iii) comparing the measured level of isotope-labeled product of the isotope-labeled metabolite in the subject to a predetermined level; wherein an increase in the measured level of isotope-labeled product compared to the predetermined level indicates efficacy of the treatment.

In another embodiment, the measured level of isotope-labeled product prior to the treatment is compared to the measured level of isotope-labeled product after the treatment, wherein an increase in the measured level of isotope-labeled product after the treatment compared to the level prior the treatment indicates efficacy of the treatment.

In one embodiment, the method could be applied to propionate oxidation disorders, including all forms of propionic acidemia, methylmalonic acidemia, cobalamin defects (cblA-J), vitamin B12 and biotin deficiency; disorders that affect hepatic mitochondrial metabolism; to test for effects of drugs that affect hepatic metabolism such as HIV medicines, and any therapies directed toward these disorders, including but not limited to, hepatic gene therapy with any vector (AAV, adenovirus, lentivirus), cell therapy, small molecules, enzyme specific chaperonins, engineered microbes/microbiome, mRNA therapy, nucleic acid therapy, enzyme replacement therapy, and genome editing therapies.

### Kits

In one embodiment, a kit for conducting isotopic breath test is provided. In certain embodiments, the kit further comprises instructions for using the kit. The instructions can be in the form of printed material or in the form of an electronic support capable of storing instructions such that they can be read by a subject, such as electronic storage media (magnetic disks, tapes and the like), optical media (CD-ROM, DVD) and the like. Alternatively or in addition, the media can contain Internet addresses that provide the instructions. The kit can be tailored for in-home use, clinical use, or research use. The kit can be tailored for in-home use, clinical use, or research use.

In one embodiment, the invention provides a kit useful for determining the efficacy of a treatment for an organic acidemia. In one embodiment, the invention provides a kit useful for determining the efficacy of a liver-directed treatment for an organic acidemia.

In one embodiment, the invention provides a kit for diagnosing a subject for an organic acidemia. The kit comprises a predetermined amount of sodium 1-¹³C propionate, a plurality of breath collection bags, guidance for testing before, during, and/or after treatment, and guidance for interpreting the test results. In one embodiment, the guidance for testing comprises instructions for directing the subject to collect breath samples at a plurality of predetermined time intervals. In one embodiment, the kit further comprises a therapeutic agent for an organic acidemia. In one embodiment, the kit further comprises guidance for discontinuing, altering, or continuing the therapeutic agent based on the test results. Additionally, the kits of the invention can contain instructions for the simultaneous, sequential or separate use of the different components contained in the kit.

In another embodiment, the kit comprises a predetermined amount of isotope-labeled metabolite, a plurality of breath collection bags, guidance for testing before, during, and/or after treatment, and guidance for interpreting the test results, wherein the isotope-labeled metabolite is 1-¹³C-propionate, 1-¹³C-glycine, or 1-¹³C-methionine.

### EXAMPLES

### Example 1: Methods and Materials

### Example 1.1 Generation of Mut^{-/-}; Tg^{INS-MCK-Mut} Mice

Mut^{-/-}; Tg^{INS-MCK-Mut} mice were created for the studies described herein. Methylmalonyl-CoA mutase (Mut) knockout mice harboring a deletion of exon three have been described, with confirmation of disrupted enzymatic function of methylmalonyl-CoA mutase and lack mRNA and protein production (Chandler et al. BMC Med Genet. 2007; 8:64, Metabolic phenotype of methylmalonic acidemia in mice and humans: the role of skeletal muscle). Mice homozygous for this mutation display neonatal lethality.

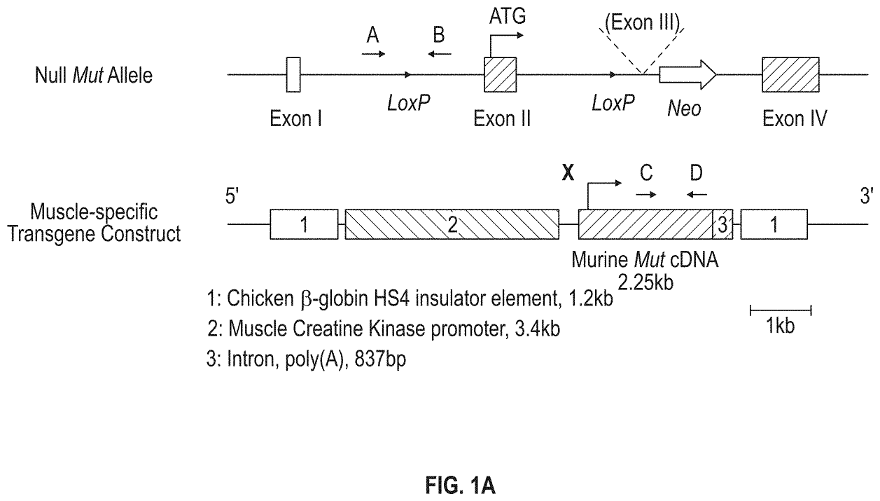

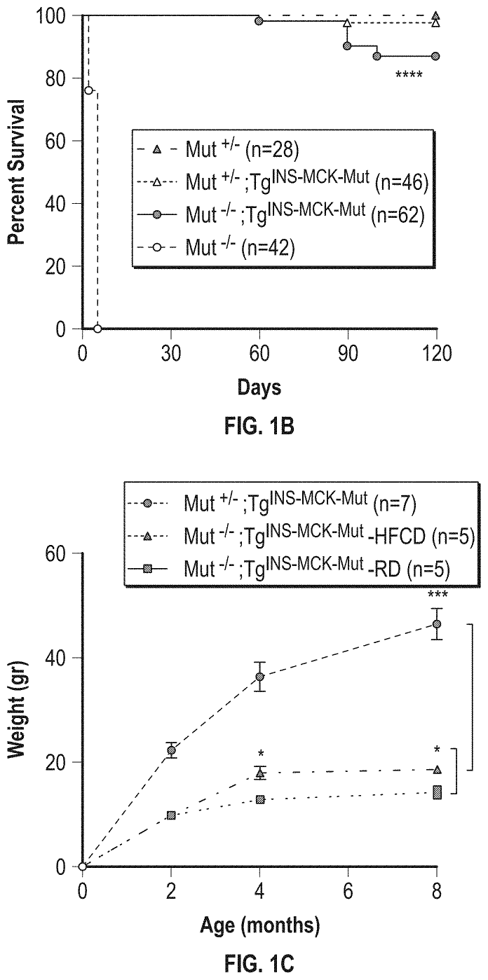

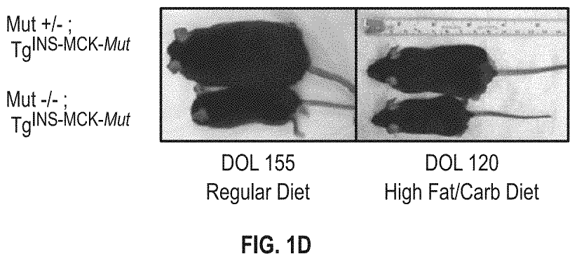

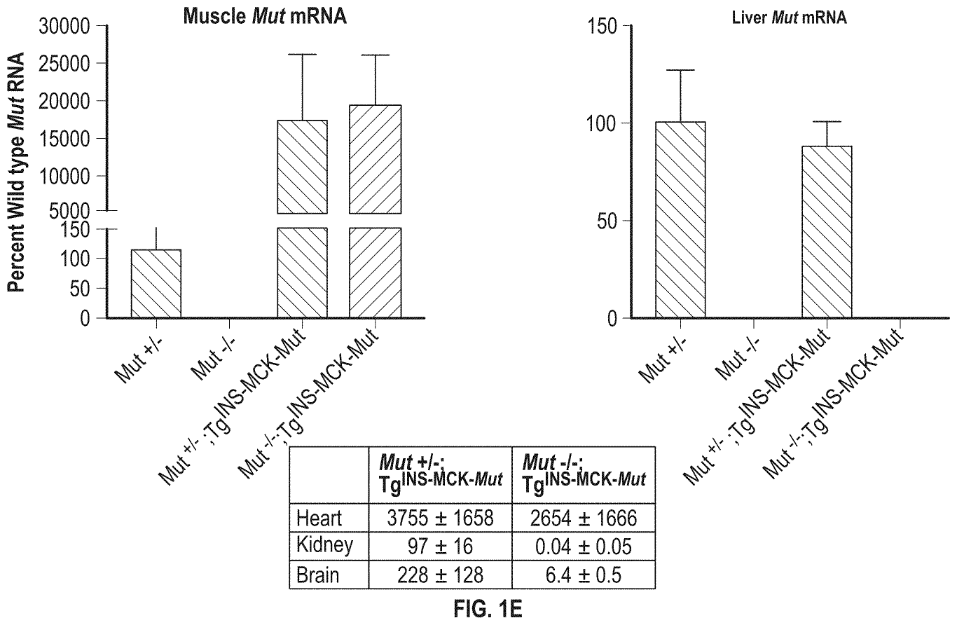

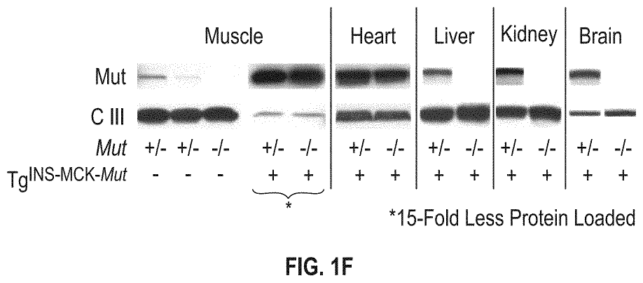

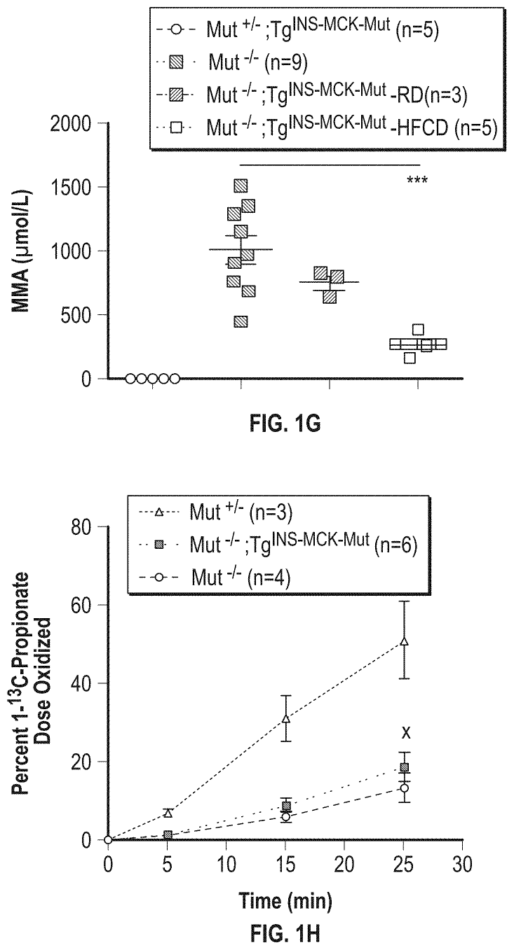

A skeletal-muscle specific transgene, Tg^{INS-MCK-Mut}, was engineered to express the murine Mut gene under the control of the muscle creatine kinase (MCK) promoter (FIG. 1A). The construct was flanked by chicken β-globin 5′ HS4 insulator elements to suppress position effect variegation (FIG. 1A). Founder C57BL/6 animals were screened for the presence of the INS-MCK-Mut transgene and bred to C57BL/6 mice to test transmission. Transgenic carrier mice were then bred with Mut^{+/-} heterozygous mice of the Mut knock-out line to generate Mut^{-/-}; Tg^{INS-MCK-Mut} mice. All animal experiments were approved by the Institutional Animal Care and Use Committee of the National Human Genome Research Institute (NHGRI), National Institutes of Health (NIH).

### Example 1.2: Mouse Genotyping

Genotyping carried out in the studies described herein. Mouse genotyping was performed on tail genomic DNA extracted using standard protocols. PCR amplifications were performed across the loxP site of the targeting construct, as well as across the Mut cDNA to detect the INS-MCK-Mut transgene. Primers used were: forward 5′-loxP site: 5′-CCATTCTGGGAAGGCTTCTA-3′ and reverse 3′-loxP site 5′-TGCACAGAGTGCTAGTTTCCA-3′. Detection of the INS-MCK-Mut transgene was completed by amplification across the Mut cDNA with primers: Forward: 5′-CATGTTGAGAGCTAAGAATC-3′ and Reverse: 5′-TAGAAGTTCATTCCAATCCC-3′.

### Example 1.3: Diet and Housing

Diet and Housing carried out in the studies described herein. Mice were housed in a controlled, pathogen-free environment with a 12 hour light/dark cycle and fed ad libitum with standard chow (PicoLab Mouse Diet 20, LabDiet, St. Louis, Mo.) or a high fat and sugar diet consisting of Diet Induced Obesity Diet (OpenSource Diets™) fruit, and Nutrical® (Tomlyn, Fort Worth, Tex.). A soft version of the regular chow (Nutra-gel diet, Bio-Serv, Flemington, N.J.) was provided for the studies involving AAV administration. For studies involving high protein diet a 70% (wt/wt) casein, or 61% protein chow, (TD.06723, Harlan Laboratories, Madison, Wis.) was provided ad libitum.

### Example 1.4: FITC-Inulin Clearance Studies

FITC-Inulin Clearance carried out in the studies described herein. Glomerular filtration rate (GFR) was assessed by the single-injection FITC-inulin clearance method. Briefly, serial plasma collections were taken from tail cuts following injection of FITC-inulin and fluorescence measurements of resultant samples were used to determine the rate of decay in comparison to standard curve. Under 1-3% isoflurane anesthesia, mice were given a single bolus retro-orbital injection of 2.5% FITC-inulin (3.74 μl/g body weight). Heparinized blood collections (5 μl volume) from tail cuts were performed at 3, 7, 10, 15, 35, 55, and 75 minutes. Plasma was separated under centrifugation (3 min, 10,000 rpm). Since pH affects FITC fluorescence values, each plasma sample was buffered by mixing 1 μl plasma with 9 μl 500 mM HEPES solution (pH 7.4). The amount of FITC label present in the samples was then measured using a fluorospectrometer at 538-nm emission (Thermo Scientific, NanoDrop 3300). A two-compartment clearance model was used to calculate GFR. Plasma fluorescence data were fit to a two-phase exponential decay curve using nonlinear regression (GraphPad Prism, GraphPad Software, San Diego, Calif.). GFR (μl/min) was calculated using the equation: GFR=I/(A/α+B/β), where I is the amount of FITC-inulin delivered by injection, A and B are the y-intercept values of the two decay rates, and α and β are the decay constants for the distribution and elimination phases, respectively.

### Example 1.5: Clinical Chemistry Screen

Clinical chemistry screen carried out in the studies described herein. Murine plasma was obtained terminally by retro-orbital blood collection using heparinized glass capillary tubes (Drummond Scientific, Broomall, Pa.) following intraperitoneal injection of pentobarbital (5 mg/ml, dose of 0.2-0.3 ml/10 g body weight). The samples were centrifuged (4° C., 10 min, 10,000 rpm), the plasma removed, and stored at −80° C. in a screw-top tube for later analysis. Methylmalonic acid was analyzed in plasma and urine samples by gas chromatography-mass spectromoetry with stable isotopic calibration.

Methylmalonic acid values were measured in patient plasma samples using liquid chromatography-tandem mass spectrometry stable isotope dilution analysis (Mayo Medical Laboratories). Estimated GFR was calculated using serum creatinine, BUN and cystatin-C, using the updated CKID equation. 24-hr urine collections were performed in a subset of patients for calculating creatinine clearance (displayed as milliliters per minute per 1.73 m2).

### Example 1.6: Western Blot & Enzymatic Activity

Western blot & enzymatic activity essay carried out in the studies described herein. Tissue samples were homogenized by tissue grinder in the presence of T-PER and Halt protease inhibitor mixture (both Pierce Biotechnology). Lysates were centrifuged at 10,000 rpm for 10 min at 4° C., and supernatants were collected. 20-30 μg of clarified protein extract were analyzed by Western blot. Protein bands were quantified using ImageJ software (NIH).

To determine mitochondrial respiratory complex activity 40-70 mg of liver tissue was homogenized in CPT (0.5 M Tris-HCl, 0.15 M KCl; pH 7.5) and centrifuged at 2,500×g for 20 min at 4° C. Resulting supernatant was used for protein quantification, detection, and enzymatic activity. 10% Extracts of CPT solution were used to measure Complex I activity by oxidation of NADH, and cytochrome c oxidase (COX or complex IV) reduction of cytochrome c at 340 and 550 nm respectively.

### Example 1.7: Histology & Immunohistochemistry

Histology & Immunohistochemistry carried out in the studies described herein. To visualize histological features and mitochondrial abnormalities, frozen sections of kidney and liver were cut and stained with COX, SDH, and combined COX-SDH reactions. These sections were examined with an Olympus BX51 microscope with a computer-assisted image analysis system. H&E staining was also performed on paraffin sections of various tissues by Histoserv, Inc, Germantown, Md.

### Example 1.8: Electron Microscopy

Electron microscopy carried out in the studies described herein. Transmission electron microscopy (EM) samples were fixed over night, embedded in resin, and cut into ˜80 nm sections and placed onto 330-mesh copper grids for staining. Samples were imaged in the JEM-1200EXII electron microscope (JEOL) at 80 kV.

### Example 1.9: Patient Studies

The human studies were approved by the NHGRI institutional review board as part of a NIH protocol (ClinicalTrials.gov identifier: NCT00078078) and were performed in compliance with the Helsinki Declaration.

### Example 1.10: Western Analysis and ELISA

Western analysis and ELISA carried out in the studies described herein. Tissue samples were homogenized with a 2-ml Tenbroeck tissue grinder (Wheaton, Millville, N.J.) in ice-cold T-PER (Pierce Biotechnology, Rockford, Ill.) in the presence of Halt protease inhibitor cocktail (Pierce Biotechnology) with deacetylase inhibitors for the post-translational modification studies. Lysates were centrifuged at 10,000 rpm for 10 min at 4° C. and supernatants were collected. Twenty to thirty micrograms of clarified protein extract were analyzed by Western blot using an affinity-purified, rabbit polyclonal antisera raised against the murine Mut enzyme at a dilution of 1:1,000. The Complex III subunit Core 2 monoclonal antibody was used as a loading control at a dilution of 1:3,000 (MS304; MitoSciences, Eugene, Oreg.). Horseradish peroxidase labeled anti-rabbit IgG (NA934VS; Amersham Biosciences, Piscataway, N.J.) or anti-mouse IgG (NA931VS; Amersham) were used as the secondary antibody at a dilution of 1:10,000 or 1:30,000, respectively. Signal was visualized using the SuperSignal West Pico chemiluminescence substrate (34080; Thermo Scientific, Rockford, Ill.).

### Example 1.11: Histology, Immunohistochemistry and Electron Microscopy

Histology, immunohistochemistry and electron microscopy carried out in the studies described herein. Tissues were fixed in 10% formalin, embedded in paraffin, sectioned, stained with hematoxylin and eosin following standard procedures (Histoserv), and examined by light microscopy. Sections of white fat, inguinal or subcutaneous were stained for UCP1 (ab-23841; Abeam) for immunohistochemistry, following the manufacturers' instructions [Ready-to-Use Vectastain Universal ABC Kit (Vector Labs)]. Tissue slides were analyzed with an Olympus microscope at a 200× magnification. Transmission electron microscopy was performed on tissues fixed at 4° C. in 2% glutaraldehyde in 0.1M cacodylate buffer (pH 7.4). The tissues were fixed with 2% OsO₄ for 2 h, washed again with 0.1M cacodylate buffer three times, subsequently washed with water and placed in 1% uranyl acetate for 1 h. The tissues were serially dehydrated in ethanol and propylene oxide and embedded in EMBed 812 resin (Electron Microscopy Sciences, Hatfield, Pa., USA). Thin sections, 80 nm thick, were obtained by utilizing an ultramicrotome (Leica, Deerfield, Ill., USA) and placed onto 300 mesh copper grids and stained with saturated uranyl acetate in 50% methanol and then with lead citrate. The grids were viewed in the JEM-1200EXII electron microscope (JEOL Ltd, Tokyo, Japan) at 80 kV and images were recorded on the XR611M, mid mounted, 10.5Mpixel, CCD camera (Advanced Microscopy Techniques Corp, Danvers, Mass., USA).

### Example 1.12: Statistical Analyses

Statistical analyses carried out in the studies described herein. All data were recorded and prepared for analysis with standard spreadsheet software (Microsoft Excel). Statistical analysis was completed using Microsoft Excel, Prism 5 (GraphPad), or IBM SPSS Version 21 statistical software. Data are presented as the means±SEM with at least three animals or subjects. When applicable, a two-tailed Student t-test or one-way ANOVA was performed followed by Bonferroni or Tukey-Kramer post hoc test for multiple comparisons. Kruskal-Wallis one-way ANOVA testing was used when groups were of different sizes. Pearson's correlation coefficient and linear regression were used to establish correlations and Kaplan-Meier analyses were performed on survival. Pearson's correlation coefficient and linear regression were employed for correlations. A P value of less than 0.05 was considered significant.

### Example 1.13: Determining Isotope-Labeled Propionate Oxidation/Metabolized Rate

The amount of ¹³CO₂ in the breath collection tubes was measured with a Europa Scientific 20/20 gas isotope ratio mass spectrometer (Europa Scientific, Crewe, UK).

The ratio of ¹³CO₂ to ¹²CO₂ (mass 45 to 44) was measured in the sample and compared to a reference gas (5% CO₂, balance 75% N2, 20% O₂). The reference gas was calibrated with international standards at three different levels of atom % ¹³C before and after each daily run to check instrument performance. The analytical precision of the instrument is 0.0001 atom % ¹³C.

The units of measurement were atom % 13C and defined as ¹³CO₂/(¹³CO2+¹²CO₂×100%.

Atom percent excess ¹³C was calculated as the difference of the atom % ¹³C from the value at time 0. APE ¹³C=atom % 13C at time (Xmin)−atom % 13C at time 0.

The atom % ¹³C values of each breath sample were used to calculate the percent of the dose recovered in the breath during each time period. The area under the curve (AUC) for each time period was calculated by the linear trapezoid method, using the atom % 13C for two consecutive points during the time period. The percent of the dose metabolized at each time point was calculated as

Total ¹³C excreted(mmol)=% ¹³C(AUC)×CO₂ production(mmol min−1)×Time(min).

The percent dose metabolized at each time point was calculated as: % dose metabolized=total ¹³C excreted (mmol)/dose administered (mmol)×100%.

### Example 2: Skeletal Muscle Expression of Mut Rescues Mut^{-/-} Mice from Neonatal Lethality and Serves as a “Metabolic Sink” for Circulating Metabolites

A construct was designed to express the murine Mut gene under the control of an insulated muscle creatine kinase (MCK) promoter (FIG. 1A). Transmitting founder lines were generated on a C57BL/6 background, C57BL/6 Tg^{INS-MCK-Mut}, and bred with C57BL/6 Mut^{+/-} mice. Mut^{-/-}; Tg^{INS-MCK-Mut} mice were born in Mendelian proportions and were protected from neonatal lethality, uniformly present in the knockout Mut^{-/-} strain on the same background, with 87% showing survival past day of life 120 (N=62; p=0.001) (FIG. 1B). The Mut^{-/-}; Tg^{INS-MCK-Mut} mice showed growth failure and remained smaller than Mut^{+/-}; Tg^{INS-MCK-Mut} littermates throughout their lifespan (FIG. 1C). Mut^{-/-}; Tg^{INS-MCK-Mut} mice on regular chow diets only achieved weights 25%-30% of their heterozygous littermates (FIG. 1C). Placing the mice on high fat and carbohydrate diets improved their survival and weight gain, though they still only achieved 40-50% of Mut^{+/-}; Tg^{INS-MCK-Mut} mice weight, who became obese on the same diet (FIG. 1D). High fat diet is frequently used to alleviate patient phenotype. At 4 months there was a significant difference between average weight on high fat (17.9±1.2 g) and regular chow (12.7±0.6 g) diets (p=0.017). Lack mRNA expression was confirmed in liver and kidney (FIG. 1 E). Abundant immunoreactive MUT was detected solely in skeletal muscle and heart of Mut^{-/-}; Tg^{INS-MCK-Mut} mice and with muscle-specific Mut RNA expression at levels comparable to protein expression (FIG. 1F).

Similar to MMA patients, methylmalonic acid levels were significantly elevated in Mut^{-/-}; Tg^{INS-MCK-Mut} mice compared with heterozygous control littermates in both high fat (p=0.001) and regular chow groups (p=0.0001). Baseline plasma MMA levels (μM) were 1107.9±66 in transgenic mice, compared to <5 in controls. Mut^{-/-}; Tg^{INS-MCK-Mut} mice on high fat diet had methylmalonic acid levels 35% of those reared on regular chow (p=0.002) (FIG. 1G).

To further assess transgene function, the in vivo oxidative capacity of Mut^{-/-}; Tg^{INS-MCK-Mut} mice was measured through detecting metabolism of numerous 1-¹³C labeled fatty acids to ¹³CO₂ via the Kreb's Cycle. Notably, the Mut^{-/-}; Tg^{INS-MCK-Mut} mice metabolized 18.4±3.6% of administered [1-¹³C]propionate dose in 25 minutes, compared with 50.7±9.8% in Mut^{+/-} and 13.1±3.7% in Mut^{-/-} (FIG. 1H).

The in vivo effects of hepatic Mut deficiency were also assessed by measuring the oxidation of 1-¹³C-methionine (FIG. 1I) and 1-¹³C-glycine (FIG. 1J). These labels reflect hepatic mitochondrial function and the activity of the glycine cleavage system, both known to be impaired in MMA patients. As predicted, the Mut^{-/-}; Tg^{INS-MCK-Mut} mice also show an impaired ability to release label when injected with these precursors.

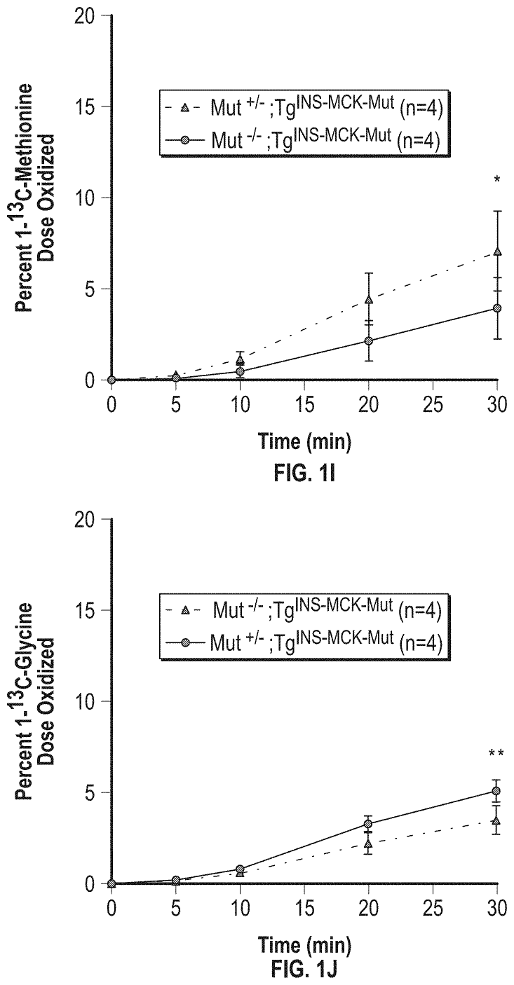

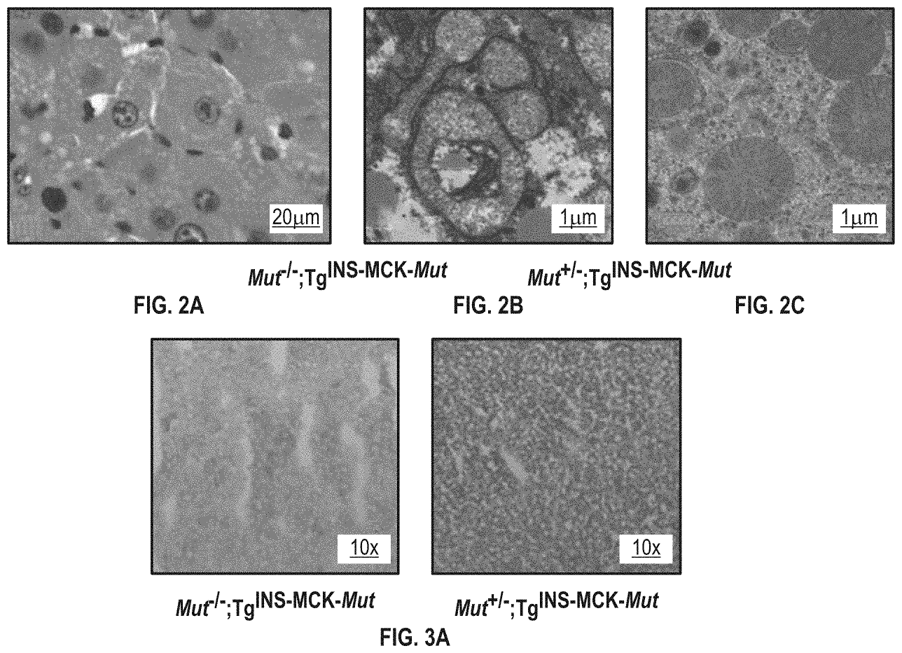

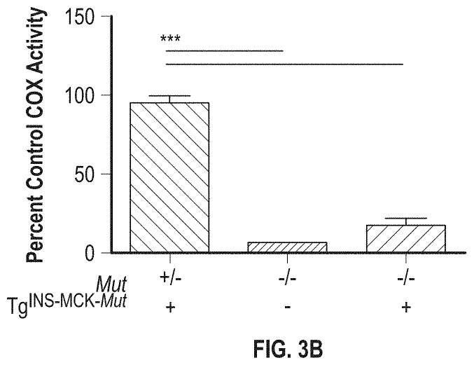

The Mut^{-/-}; Tg^{INS-MCK-Mut} animals developed significant liver pathology, characterized by severe diffuse lipidosis, vacuolization of the cytoplasm, and megamitochondria formation, which was associated with decreased respiratory chain complex IV activity (18.2±7.4% relative to controls), similar to the Mut^{-/-} mice (FIG. 2A). Further, electron microscopy of Mut^{-/-}; Tg^{INS-MCK-Mut} livers showed mitochondria that are enlarged with shortened and flattened or no cristae (FIG. 2B). Other mitochondria formed a rosette-like pattern that may represent autophagy or mitophagy. These findings resemble changes previously noted in electron microscopy of an MMA patient liver. Control littermates had normal hepatic ultrastructure (FIG. 3C).

Cytochrome oxidase (COX) and succinic dehydrogenase (SDH) were both depleted in Mut^{-/-}; Tg^{INS-MCK-Mut} mice compared with heterozygous littermates, indicating diminished electron transport chain activity and mitochondrial biogenesis (FIG. 3 A,B).

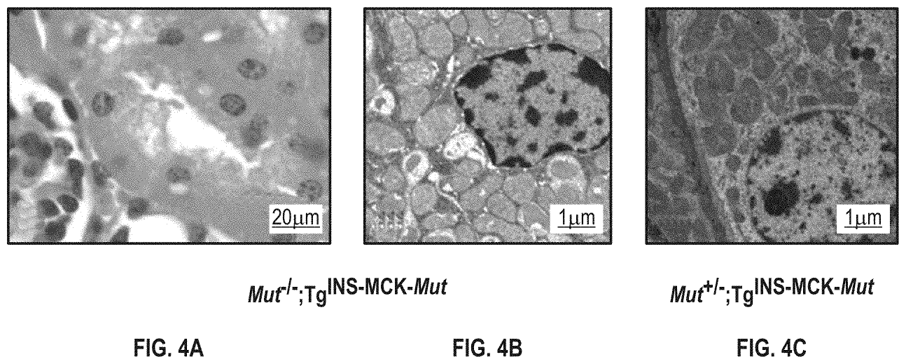

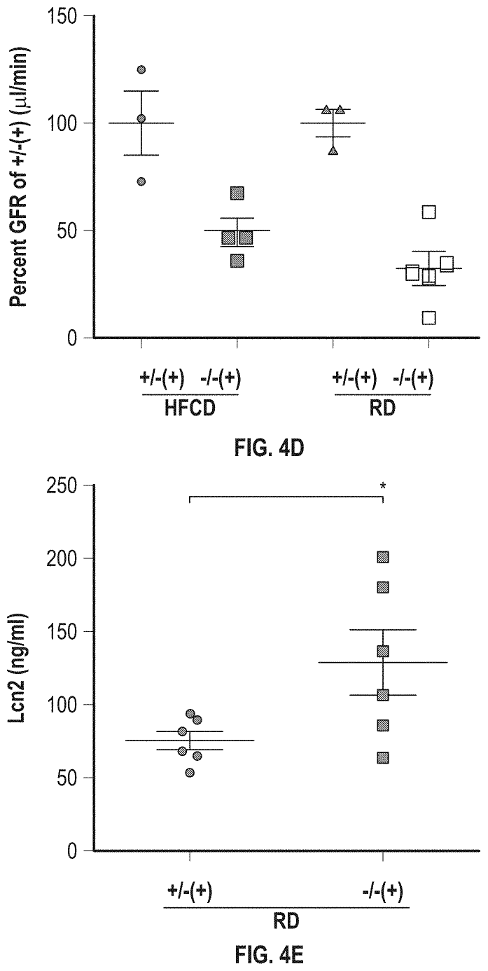

H&E staining showed that Mut^{-/-}; Tg^{INS-MCK-Mut} mice kidneys contain large, eosinophilic vacuoles in their proximal tubules (FIG. 4A) and megamitochondria (FIG. 4B). These changes were similar to those seen in MMA patient kidneys (Manoli et al, PNAS, 2013, 13552-13557, Targeting proximal tubule mitochondrial dysfunction attenuates the renal disease of methylmalonic acidemia) but not control littermates (FIG. 4C). Glomerular filtration rate (GFR) measurements, performed in vivo with FITC-inulin, showed that Mut^{-/-}; Tg^{INS-MCK-Mut} mice had 49% filtration compared with Mut^{+/-}; Tg^{INS-MCK-Mut} mice (p=0.02) on high fat diet (FIG. 4D). Similarly on the regular chow, Mut^{-/-}; Tg^{INS-MCK-Mut} mice had a filtration rate 32% of that of Mut^{+/-}; Tg^{INS-MCK-Mut} mice (p=0.001). GFR measurements between Mut^{-/-}; Tg^{INS-MCK-Mut} mice on high fat and regular diets showed no statistical difference (p=0.15) (FIG. 4D). A kidney disease biomarker, lipocalin 2, was measured, as described in prior work (Manoli et al, PNAS, 2013, 13552-13557, Targeting proximal tubule mitochondrial dysfunction attenuates the renal disease of methylmalonic acidemia) and validated in a large MMA patient cohort. Plasma Lcn2 concentrations were significantly elevated in the Mut^{-/-}; Tg^{INS-MCK-Mut} mice compared to their heterozygote littermates (p=0.04), and correlated with the GFR measurements (FIG. 4E), further validating the reduced GFR measured and the validity of Lcn2 as a renal biomarker in MMA.

Selective muscle expression of the Mut enzyme by transgenesis at levels matching or exceeding the heterozygous controls in the skeletal and cardiac muscle resulted in near uniform rescue of the neonatal lethal phenotype of the Mut^{-/-} mice, but was unable to prevent liver and kidney damage.

Severe hepatorenal pathological changes in the Mut^{-/-}; Tg^{INS-MCK-Mut} animals replicate the hepatic and renal pathology seen in MMA patients.

### Example 3: Isotope Oxidation Results in MCK Mouse Model

In Vivo Stable Isotope Oxidation Studies: Stable isotope studies were performed in 4 Mut−/−; Tg^{INS-MCK-Mut} and 4 control littermates. Closed circuit, constant volume respiratory chambers were used to collect and measure enrichment of ¹³CO₂ in mice, as described previously (Chandler and Venditti, 2009 and 2010, Manoli et al 2013). Mice received IP injections with tracer amounts (10 μl/g of [10 mg/ml] tracer solution) of 99.9% ¹³C-isotopomers. Aliquots of air were removed every 5-10 min, while CO₂ was continuously monitored. ¹³CO₂ enrichment was measured by isotope ratio mass spectroscopy (Metabolic Solutions, Nashua, N.H.). Results were reported as delta: δ=(¹³C:¹²Csample/¹³C:¹²Cstandard-1)*1000, where Delta C¹³ units=per mil (‰)=molecules per thousand more than in the standard. Cumulative percentage of total isotopomer dose metabolized was subsequently calculated, using the formula: Percentage of dose metabolized=total ¹³C excreted [mmol/dose (mmol)×100%].

The following 1-C labeled stable isotopes were used: ¹³C Sodium Propionate, ¹³C-Methionine, ¹³C-Glycine, ¹³C-Pyruvate, ¹³C-Octanoate, ¹³C-α-Ketoisocaproic Acid, ¹³C-Leucine, ¹³C-Acetate, ¹³C-Phenylalanine. Stable isotopomers were purchased from Cambridge Isotope Laboratories. Variables were compared for each substrate and time point using a two-sided unpaired t-test and considered statistically significant at p<0.05. The results are shown in Table 1. The oxidation of labels primarily affected by the Mut enzymatic deficiency (1-¹³C-propionate), reflective of perturbed hepatic mitochondrial metabolism (1-¹³C-methionine) or impaired hepatic activity of the glycine cleavage pathway (1-¹³C-glycine), was substantially reduced in the Mut^{-/-}, Tg^{INS-MCK-Mut} mice, compared to labels that require mitochondrial metabolism (1-¹³C-acetate) or that reflect the bicarbonate space (1-¹³C-bicarbonate). The results show the labels probe aspects of in vivo metabolism in a disease-related fashion and are not reflective of a generalized mitochondrial effect or acidosis.

TABLE 1

| | Mut^{-/-} Oxidation Rate | Mut^{+/-} Oxidation Rate | |
|---|---|---|---|
| Isotopomer | (μmol/g/hour) | (μmol/g/hour) | p-value, t-test |
| 1-¹³C-propionate | 29.04 +/− 21.80 (n = 7) | 83.52 +/− 13.16 (n = 7) | p < 0.005 |
| 1-¹³C-glycine | 0.07 +/− 0.007 (n = 4) | 0.14 +/− 0.003 (n = 3) | p < 0.0001 |
| 1-¹³C-methionine | 6.5 +/− 2.5 (n = 4) | 11.5 +/− 3.4 (n = 4) | p < 0.05 |
| 1-¹³C-bicarbonate | 122.7 +/− 29.9 (n = 5) | 77.7 +/− 47.1 (n = 5) | N.S. |
| 1-¹³C-acetate | 88.2 +/− 26.5 (n = 5) | 69.5 +/− 17.7 (n = 5) | N.S. |

### Example 4: 1-¹³C-Propionate Oxidation Predicts the Phenotypic and Metabolic Response to AAV Gene Therapy for MMA

An AAV gene therapy vector was prepared. In brief, the mouse Mut cDNA was cloned in between AAV2 ITRs, and under the control of the enhanced chicken beta actin promoter (CBA) and packaged using a serotype 9 capsid as previously described (Sénac J S, et al. Gene therapy in a murine model of methylmalonic acidemia using rAAV9-mediated gene delivery. Gene Ther. 2012 April; 19(4):385-91.). Next, a group of Mut^{-/-}; Tg^{INS-MCK-Mut} mice were injected with a dose of 2.5 GC/kg AAV by the retro-orbital route. Mut^{-/-}; Tg^{INS-MCK-Mut} mice are growth retarded (FIG. 1C), as indicated by being underweight compared to their littermates, displaying very high levels of methylmalonic acid (FIG. 1G) and an impaired ability to oxidize 1-¹³C-propionic acid (FIG. 1H).

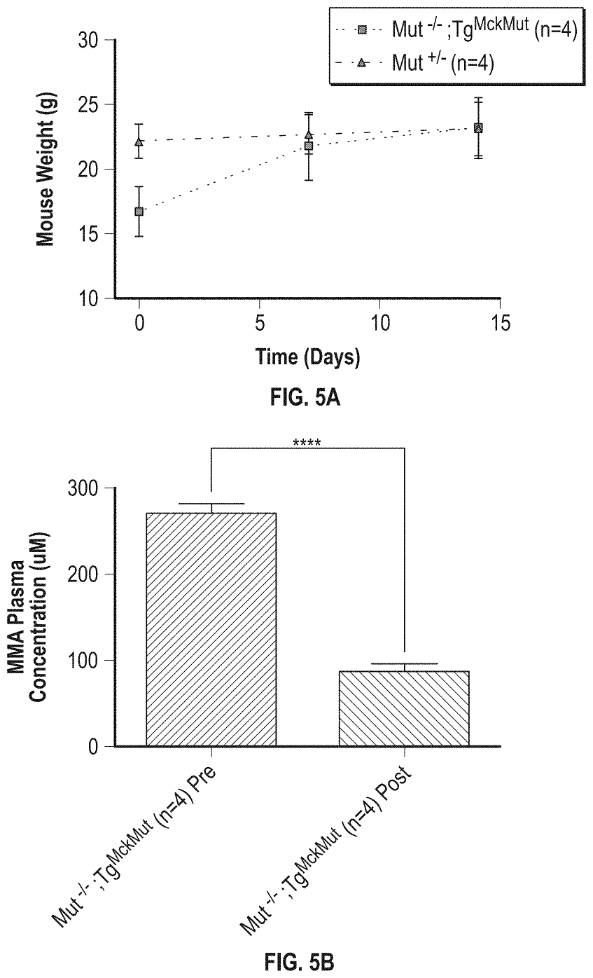

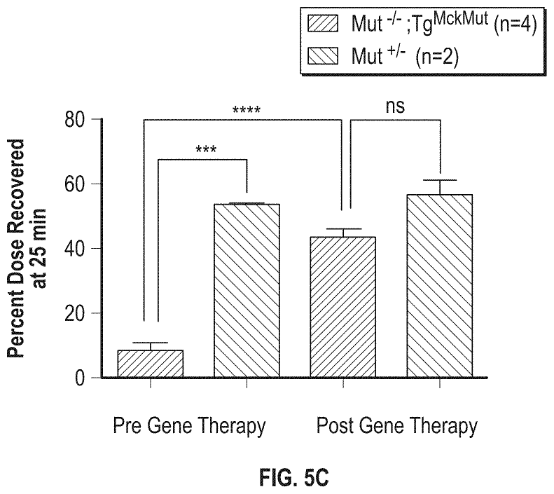

After receiving AAV9 CBA Mut gene therapy, the Mut^{-/-}; Tg^{INS-MCK-Mut} mice showed a remarkable improvement in weight, achieving the size of unaffected control littermates in 2 weeks (FIG. 5A). A substantial metabolic improvement was also noted, with serum methylmalonic acid decreasing almost 3-fold (FIG. 5B).

In these same mice, the ability to oxidize 1-¹³C-propionic acid after gene therapy was nearly restored to control levels (FIG. 5C), despite the fact that the serum methylmalonic acid was elevated, demonstrating the utility of 1-¹³C-propionate oxidation as an in vivo assay for Mut activity.

Systemic mRNA therapy for MMA was also evaluated in Mut^{-/-}; Tg^{INS-MCK-Mut} mice in an effort to validate the oxidative measurement disclosed herein in an animal model of the disease. Metabolic improvement in the form of decreased serum methylmalonic acid, hepatic response in the form of increased expression of methylmalonyl-CoA mutase (MUT), and oxidative response in the form of augmented ability to oxidize isotope-labeled propionate were observed. Oxidation of the isotope-labeled propionate correlated well with the metabolic and hepatic responses, indicating that the oxidative measurement constitutes a valid in vivo assay of efficacy of treatment for MMA.

### Example 5: Patient Studies of Isotope Oxidation

An open-circuit indirect calorimetry method (ventilated hood) was used to measure basal or resting energy expenditure in subjects of various ages and sizes. A metabolic cart (ParvoMedics TrueOne2400 Sandy, Utah) was used to measure subjects' O₂ consumption and CO₂ production at supine posture for 30-45 minutes. The flow rate of the open-circuit system was set between 20-30 L/min to achieve 0.9-1.2% end-tidal CO₂ concentration, which is the optimal sensitivity range for the near-infrared CO₂ analyzer and with minimal impact on subject's normal breathing patterns (inspired CO₂ concentration higher than 3% could cause hyperventilation, headaches, and nausea).

To measure the in vivo oxidative capacity for 1-¹³C propionate in methylmalonic acidemia patients and healthy volunteers, a single bolus of sodium 1-¹³C-propionate was delivered by mouth or via a gastrostomy tube over no more than 2 min, followed by a similar amount of water consumption. All participants were fasting for 3 hours prior and throughout the 1st hour of the procedure, but were allowed access to water for p.o. or g-tube fluid intake, if desired. Food was offered after the first hour of breath sampling, if clinically indicated.

The dose administered was 0.5 mg/kg dissolved in sterile water at a concentration of 1 mg/ml (99 atom % 13C, clinical grade, MW: 97.05 g/mol; from Cambridge Isotope Laboratories Andover, Mass., prepared on the day of the study by the NIH Pharmaceutical Development Service for human use). Subsequently, serial breath samples were obtained 2, 5, 10, 15, 20, 25, 30, 40, 50, and 60 minutes after isotope administration. 10 ml aliquots of expired breath were collected with disposable breath collection kits (EasySampler™ Breath Test Kit, Quintron) into vacutainers before isotope administration and at structured time points over 2 hours (1, 5, 7, 10, 15, 20, 25, 30, 45, 60, 90, and 120 minutes), for measurement of 13CO₂.

The isotope ratio (¹³C/¹²C) of the expired gas was measured by a gas isotope ratio mass spectrometer (Metabolic Solutions Inc., Nashua, N.H.). Results were reported as delta: δ=(¹³C:¹²:¹²Cₛₐₘₚₗₑ/¹³C:¹²C_{standard}−1)*1000. APE: Atomic percent excess: the level of isotopic abundance above a given background reading, which is considered zero. Percent Dose Oxidized at each time point=CO₂ production rate×Σ(APE (t)/(mmol C¹³ administered)×100, where CO₂ production rate was the one measured by the indirect calorimetry on the same day just prior to the isotope study.

The parallel study of the oxidation of 1-¹³C-acetate into bicarbonate and exhaled ¹³CO₂ was performed in selected patients, as an independent assessment of the activity of the Krebs cycle. This was used to calculate the acetate recovery factor and allowed for a more accurate estimate of the 1-¹³C-propionate oxidation rate.

The test cohort was comprised of 41 patients with MMA (26 mut0, aged 3-37 years, 6 mut-, aged 9-30 years, and 9 cblA, aged 4-41 years), 8 healthy volunteers, and 8 heterozygote parents of affected individuals. Within the affected group, 12 individuals had previously received a liver (LT), kidney (KT), combined liver-kidney (LKT), or partial (graft) liver-kidney (pLKT) transplant (2 LT, 3 KT, 6 LKT, 1 pLKT).

A metabolic cart (ParvoMedics TrueOne2400 Sandy, Utah) was used to measure subject O₂ consumption (V_{O2}), CO₂ production (V_{CO2}), and resting energy expenditure (REE) at supine posture for 25-35 minutes. Seven of the healthy volunteers were tested three times over a two-month period to evaluate inter- and intra-individual variability.

Sodium 1-¹³C-propionate (Cambridge Isotope Laboratories, Andover, Mass.) was prepared for human use. A dose of 0.5 mg/kg (or 0.5 ml/kg) body weight (BW) was administered to the study subjects orally or through a G-tube as a bolus over no more than 2 minutes, followed by similar amount of water consumption. Breath samples were collected serially via disposable breath collection kits (EasySampler™ Breath Test Kit, Quintron) prior to isotope administration, and at specified time points over 2 hours (1, 5, 7, 10, 15, 20, 25, 30, 45, 60, 90, and 120 minutes). Vital signs were taken prior to and 10 minutes after isotope consumption to ensure that the compound was tolerated without any adverse event. Measurements of the isotopic ratio (¹³C/¹²C) in expired gas were determined by isotope ratio mass spectrometry (Metabolic Solutions, Nashua, N.H.).

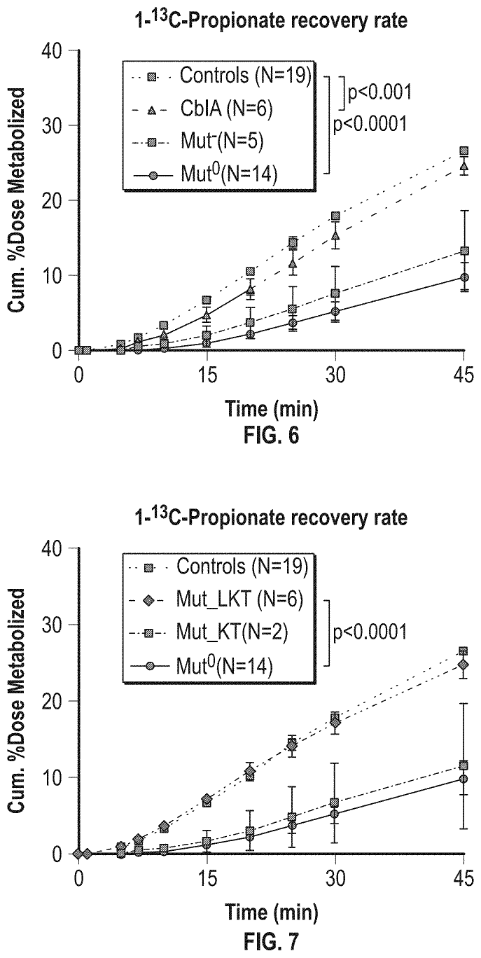

Decreased propionate oxidation was observed in all MMA patients vs. controls (p<0.0001) (FIG. 6). The most severe patients, mut0 (n=16) and mut-(n=6), showed almost no movement of label, while more mildly affected patients with cblA MMA had substantial oxidation of 1-¹³C-propionate.

Combined liver-kidney transplant (LKT) (n=3) and liver transplant (LT) (n=1) recipients showed a complete restoration of oxidation rates to control levels, while kidney transplant (KT) (n=2) recipients were not significantly different than non-transplanted patients (p<0.0001 compared to controls, not significantly different than mut0/−) (FIG. 7). While cblA B12-responsive patients showed improved activity compared to the mut cohort, propionate oxidation was significantly impaired relative to controls (P<0.0016).

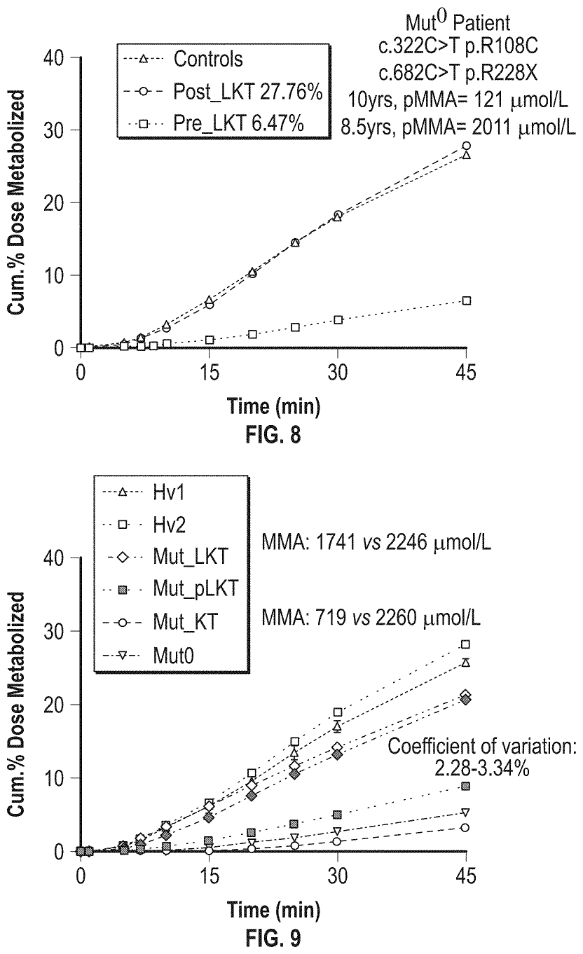

A drastic improvement in propionate oxidation was noted in a patient who underwent a combined liver kidney transplantation. Before the procedure, the oxidation rate was 6.5%, while after, it normalized to the level seen in healthy controls (FIG. 8).

Reproducibility was established with repeat testing; 10 repeat tests were obtained in 8 MMA patients; results from two mut0 patients, one LKT patient, one auxiliary liver allograft post KT recipient, and one KT patient are shown in FIG. 9. The results are reproducible for administering the composition having sodium 1-¹³C-propionate via oral or gastric route. The results from oral and gastric routes are comparable.

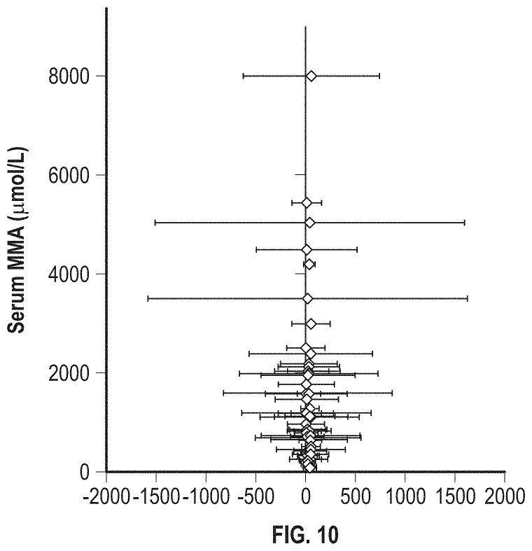

In contrast, the coefficient of variability for the measurement of serum methylmalonic acid in MMA patients was much greater, ranging from 1.3-77%, as presented in FIG. 10.

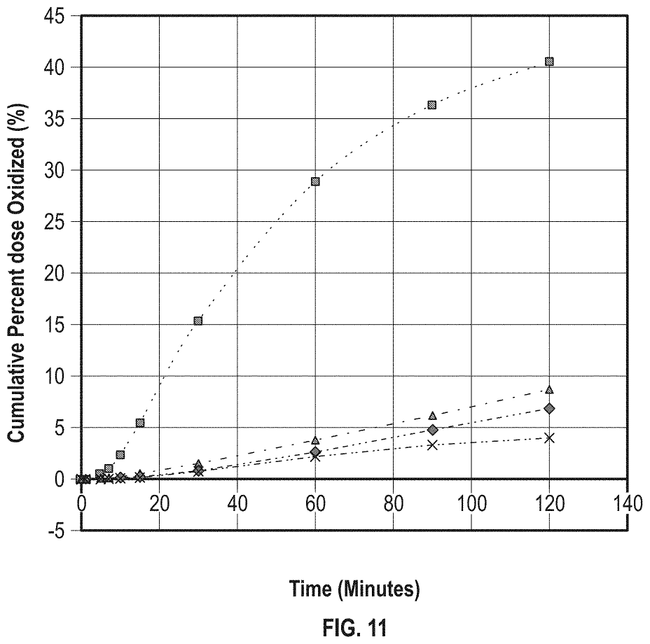

An extension of the afore-mentioned and described method of measuring 1-¹³C-propionate oxidation in human subjects was next applied to patients with the related disorder, propionic acidemia. As seen in FIG. 11, more severe patients have a diminished ability to metabolize 1-¹³C-propionate. Accordingly, metabolism of 1-¹³C-propionate may be used to determine the severity of PA in the patients.

In yet another embodiment, the metabolism of 1-¹³C-isotopomers for monitoring of therapeutic interventions for other related metabolic disorders is provided. In these examples, tracers are administered PO or IV and the measurements of the isotopic ratio (¹³C/¹²C) in expired gas is obtained by either measurement using IRMS or the Breath-ID platform, or other method to measure ¹³C/¹²C CO₂ enrichment. Many metabolic disorders where hepatic metabolism of the tracer into CO₂, representing substrate oxidation, are candidates for non-invasive isotopic monitoring to ascertain efficacy of therapeutic intervention which might include liver directed gene therapy using AAV vectors, enzyme replacement therapy, genome editing, mRNA therapy, microbiome manipulations, chaperones, small molecule activators, and cofactors. Table 2 lists examples of the disorders, labels, and dosing.

TABLE 2

| | | Dose Ranges and |
|---|---|---|
| Disorder | Label | Routes |
| Maple Syrup Urine | 1-¹³C leucine | 0.1-5 mg/kg; PO; IV |
| Disease (MSUD) | 1-¹³C isoleucine | |
| | 1-¹³C valine | |
| | 1-¹³C -alphaketoisocaproic | |
| | acid | |
| Phenylketonuria (PKU) | 1-¹³C -phenylalanine | 0.1-5 mg/kg; PO; IV |
| Biopterin recycling | 1-¹³C -phenylalanine | 0.1-5 mg/kg; PO; IV |
| defects | | |
| Fatty acid oxidation | 1-¹³C -octanoate | 0.1-5 mg/kg; PO; IV |
| disorders (FAOD), | | |
| medium chain (MCAD) | | |
| Fatty acid oxidation | 1-¹³C -palmitate | 0.1-5 mg/kg; PO; IV |
| disorders (FOAD), long | | |
| chain (LCAD) | | |
| Fatty acid oxidation | 1-¹³C -palmitate | 0.1-5 mg/kg; PO; IV |
| disorders (FOAD), very | 1-¹³C -octanoate | |
| long chain (VLCAD) | | |
| Fatty acid oxidation | 1-¹³C -palmitate | 0.1-5 mg/kg; PO; IV |
| disorders, long chain | | |
| hydroxylacyl-CoA | | |
| dehydrogenase (TFP, | | |
| LCHAD) | | |
| Glyogen storage | 1-¹³C -glucose | 0.1-5 mg/kg; PO; IV |
| disorders (GSD1,3) | | |
| Peroxisomal disorders | 1-¹³C -docosahexaenoic acid | 0.1-5 mg/kg; PO; IV |
| | 1-¹³C -phytanic acid | |
| Multiple Acyl-CoA | 1-¹³C -palmitate | 0.1-5 mg/kg; PO; IV |
| dehydrogenase | 1-¹³C -octanoate | |
| deficiency (MADD) | 1-¹³C -lysine | |
| | 1-¹³C -tryptophan | |
| Mitochondrial disorders, | 1-¹³C -methionine | 0.1-5 mg/kg; PO; IV |
| including complex 1, | | |
| 2, 3 and 4 | | |
| Mitochondrial disorders, | 1-¹³C -methionine | 0.1-5 mg/kg; PO; IV |
| DNA depletion syndromes | | |
| Pyruvate dehydrogenase | 1-¹³C -pyruvate | 0.1-5 mg/kg; PO; IV |
| deficiency | | |
| Krebs cycle enzyme | 1-¹³C -acetate | 0.1-5 mg/kg; PO; IV |
| defects | | |
| Non-ketotic | 1- C 13-glycine | 0.1-5 mg/kg; PO; IV |
| hyperglycinemia | | |
| Non-alcoholic liver | 1-¹³C -methionine | 0.1-5 mg/kg; PO; IV |
| disease | 1-¹³C -alphaketoisocaproic | |
| | acid | |
| Galactosemia (GALT) | 1-¹³C -galactose | 0.1-5 mg/kg; PO; IV |
| Tyrosinemia | 1-¹³C -tyrosine | 0.1-5 mg/kg; PO; IV |
| Glutaric acidemia type I | 1-¹³C -lysine | 0.1-5 mg/kg; PO; IV |
| (GCDH) | 1-¹³C -tryptophan | |
| Isovaleric acidemia | 1-¹³C -leucine | 0.1-5 mg/kg; PO; IV |
| | 1-¹³C -alphaketoisocaproic | |
| | acid | |
| Fatty acid oxidation | 1-¹³C -palmitate | 0.1-5 mg/kg; PO; IV |
| disorder, Carnitine | 1-¹³C -octanoate | |
| transport and | | |
| metabolism (CPT1, | | |
| CPT2, CTD) | | |
| 3-MCC deficiency | 1-¹³C -leucine | 0.1-5 mg/kg; PO; IV |
| | 1-¹³C -alphaketoisocaproic | |
| | acid | |
| Liver disease, NASH | 1-¹³C -methionine | 0.1-5 mg/kg; PO; IV |
| and mitochondrial | | |
| | 1-¹³C -alphaketoisocaproic | |
| | acid | |
| Organic acidemias, | 1-¹³C -propionate | 0.1-5 mg/kg; PO; IV |
| MMA and PA | 1-¹³C -methionine | |
| | 1-¹³C -glycine | |

In one embodiment, the disorder is classical phenylketonuria (PKU) or a biopterin cofactor disorder, the label is 1-¹³C-phenylalanine, the dose ranges are 0.1-5 mg/kg, and the route is PO or IV. In another embodiment, the disorder is maple syrup urine disease (MSUD) and the labels are either 1-¹³C-leucine, 1-¹³C-isoleucine, 1-¹³C valine or 1-¹³C-alphaketoisocaproic acid; the dose ranges are 0.1-5 mg/kg, and the route is PO or IV. In another embodiment, the disorder is a medium chain fatty acid oxidation disorder such as medium chain acylCoA dehydrogenase deficiency, the label is 1-¹³C-octanoate, the dose ranges are 0.1-5 mg/kg, and the route is PO or IV. In another embodiment, the disorder is another fatty acid oxidation disorder such as very long chain acylcoA dehydrogenase deficiency, the long chain hydroxylacylcoa dehydrogenase deficiency, the trifunctional protein deficiency, a carnitine metabolic disorder such as carnitine palmitoyl transferase type 1 or 2 deficiency or the carnitine transporter disorder, the label is 1-¹³C-palmitate, the dose ranges are 0.1-5 mg/kg, and the route is PO or IV. In another embodiment, the disorder is a glycogen storage disorder, such as GSD type 1 or GSD type 3, the label is 1-¹³C-glucose, the dose ranges are 0.1-5 mg/kg, and the route is PO or IV. In another embodiment, the disorder is Multiple Acyl-CoA dehydrogenase deficiency (MADD), the labels are either 1-¹³C-palmitate, 1-¹³C-octanoate, 1-¹³C-lysine, or 1-¹³C-tryptophan, the dose ranges are 0.1-5 mg/kg, and the route is PO or IV. In another embodiment, the disorders are mitochondrial disorders, including complex 1, 2, 3 and 4 deficiencies or mitochondrial DNA depletion syndromes, the label is 1-¹³C-methionine, the dose ranges are 0.1-5 mg/kg, and the route is PO or IV. In another embodiment, the disorder is the pyruvate dehydrogenase deficiency (PDH), the label is 1-¹³C-pyruvate, the dose ranges are 0.1-5 mg/kg, and the route is PO or IV. In another embodiment, the disorders are Krebs cycle enzyme defects, the label is 1-¹³C-acetate, the dose ranges are 0.1-5 mg/kg, and the route is PO or IV. In another embodiment, the disorders are the non-ketotic hyperglycinemias (NKH), the label is 1-¹³C-glycine, the dose ranges are 0.1-5 mg/kg, and the route is PO or IV. In another embodiment, the disorder is galactosemia, the label is 1-¹³C-galactose, the dose ranges are 0.1-5 mg/kg, and the route is PO or IV. In another embodiment, the disorder is tyrosinemia type I, II or III, the label is 1-¹³C-tyrosine, the dose ranges are 0.1-5 mg/kg, and the route is PO or IV. In another embodiment, the disorder is glutaric acidemia type 1 (GA1), the labels are 1-¹³C-lysine or 1-¹³C-tryptophan, the dose ranges are 0.1-5 mg/kg, and the route is PO or IV. In another embodiment, the disorder is isovaleric acidemia (IVA), the label is 1-¹³C-leucine or 1-¹³C-alphaketoisocaproic acid, the dose ranges are 0.1-5 mg/kg, and the route is PO or IV. In another embodiment, the disorder is 3-methylcrotonylcoa carboxylase deficiency, the label is 1-¹³C-leucine or 1-¹³C-alphaketoisocaproic acid, the dose ranges are 0.1-5 mg/kg, and the route is PO or IV. In another embodiment, the disorders are organic acidemias such as methylmalonic acidemia or propionic acidemia (MMA, PA), the labels are 1-¹³C-propionate, 1-¹³C-methionine, or 1-¹³C-glycine, the dose ranges are 0.1-5 mg/kg, and the route is PO or IV. In yet another embodiment, liver diseases such as non-alchoholic steatohepatitis (NASH) can be diagnosed and monitored by the oxidation of 1-¹³C-methionine or 1-¹³C-alphaketoisocaproic acid, the dose ranges are 0.1-5 mg/kg, and the route is PO or IV.

The results of oxidation measurements in mice are presented in Table 3. These studies were conducted using N=5 mice with MMA (Mut^{-/-}; Tg^{INS-MCK-Mut} mutants) and N=5 sex matched littermates (controls). Each mouse was injected with the labels depicted in Table 3 and the oxidation rates and kinetics of ¹³C/¹²C CO₂ enrichment were analyzed.

TABLE 3
Isotopomer oxidation rates in MMA (Mut^{-/-};Tg^{INS-MCK-Mut}) mice

| | | Cumulative % of total dose metabolized | | |
|---|---|---|---|---|
| | | Mutants, | Controls, | t-test |
| Isotopomer | Time-point | mean (SD) | mean (SD) | p-value |
| Sodium 1-¹³C-Acetate | 25′ | 42.23 (14.32) | 38.02 (11.79) | 0.33 |
| Sodium 1-¹³C-Pyruvate | 25′ | 52.99 (9.85) | 52.16 (17.06) | 0.47 |
| Sodium 1-¹³C-Octanoate | 30′ | 44.71 (8.58) | 31.94 (11.58) | 0.06 |
| Sodium | 25′ | 24.00 (12.95) | 33.95 (3.66) | 0.10 |
| 1-¹³C-α-Ketoisocaproic | | | | |
| Acid | | | | |
| L-1-¹³C-Leucine | 25′ | 12.44 (3.10) | 13.54 (1.50) | 0.27 |
| L-2-¹³C-Leucine | 25′ | 3.32 (0.85) | 3.68 (2.34) | 0.39 |
| 2-¹³C-Valine | 25′ | 5.15 (2.19) | 4.41 (2.63) | 0.34 |
| L-1-¹³C-Phenylalanine | 25′ | 5.71 (2.74) | 10.75 (7.38) | 0.12 |

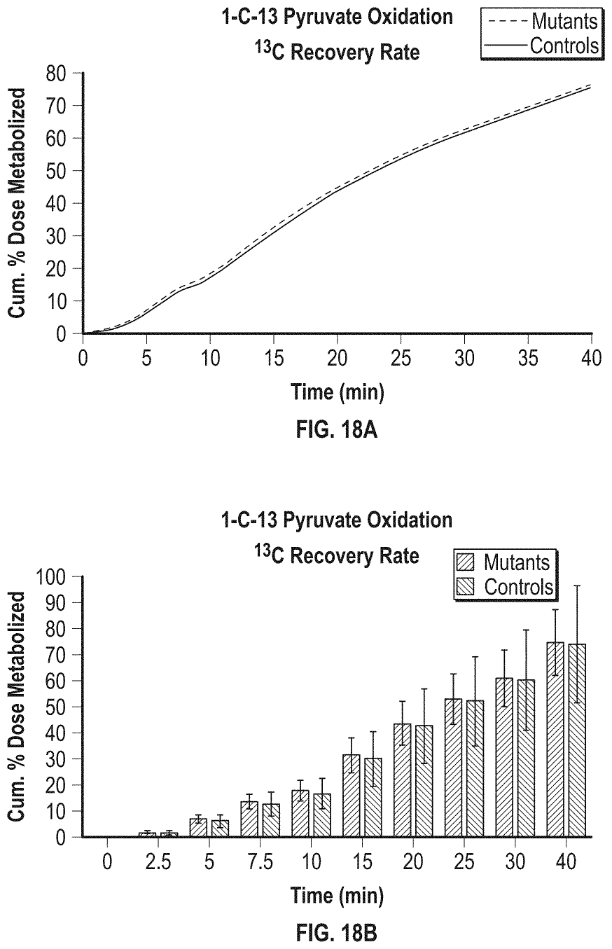

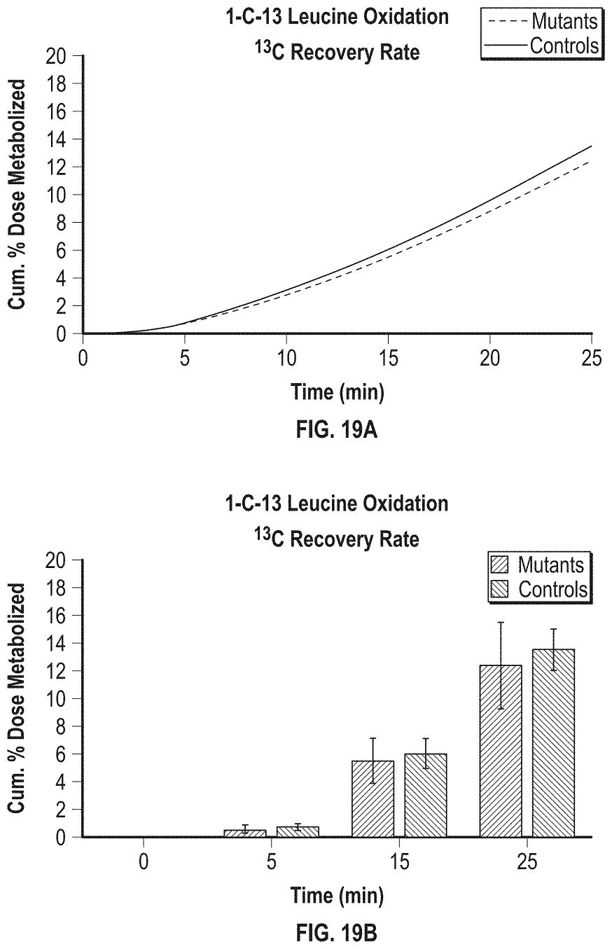

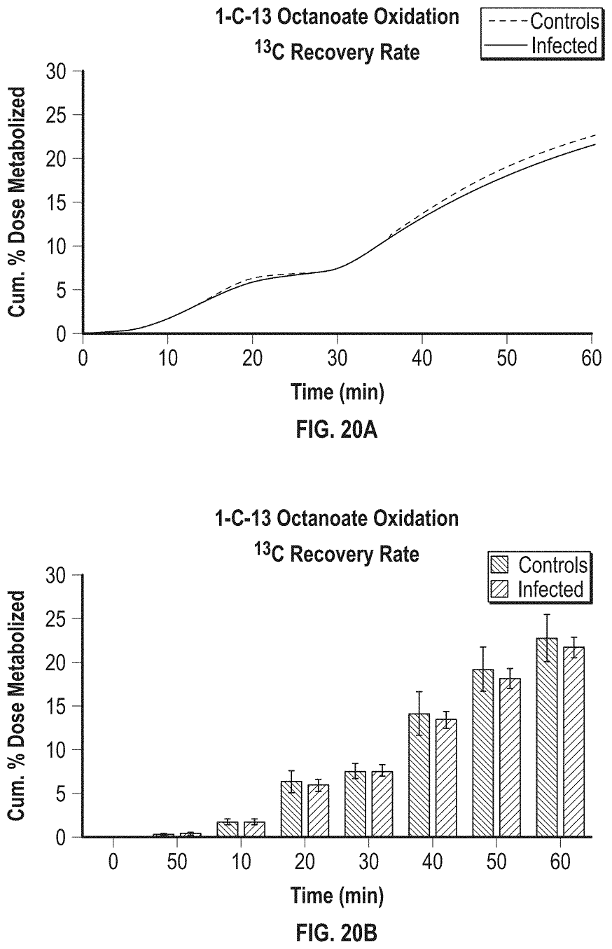

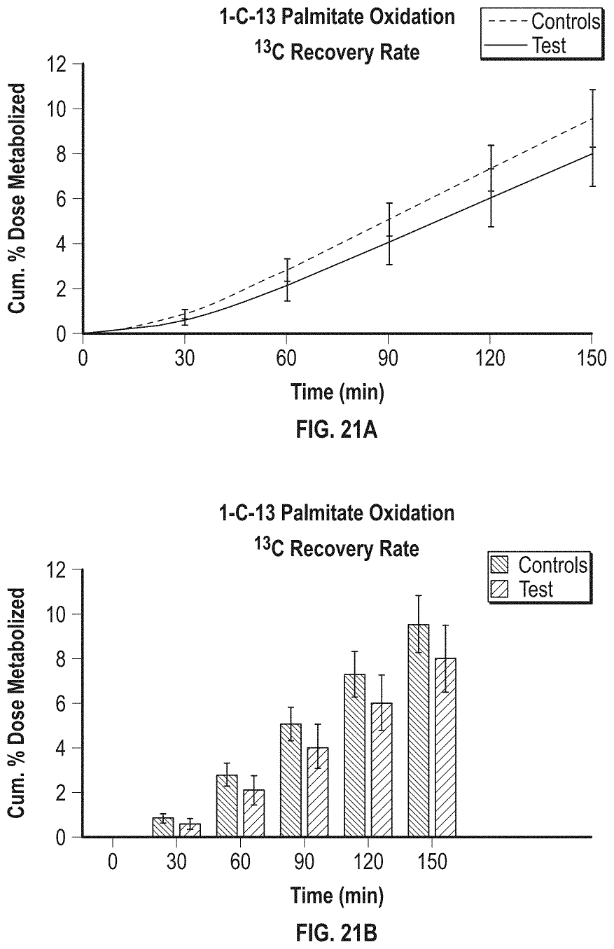

A series of representative studies showing 1-¹³C-pyruvate (FIGS. 18A-B),-leucine (FIGS. 19A-B), -octanoate (FIGS. 20A-B), and -palmitate (FIGS. 21A-B) recovery rates over time is presented.

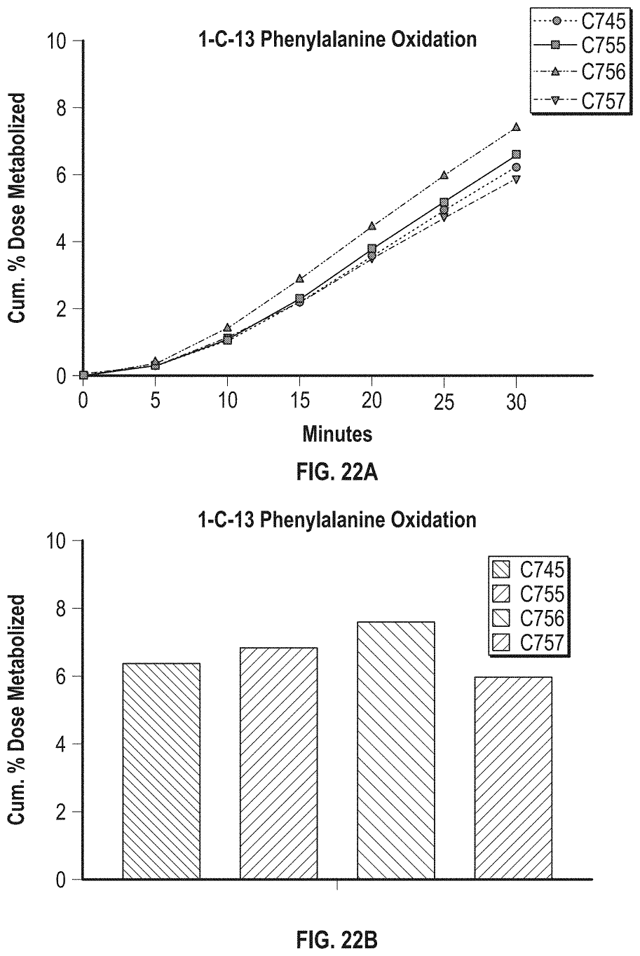

The last set of studies details the oxidation of 1-C-13 phenylalanine (FIGS. 22A-B) in normal mice.

### Example 6: Stable Isotope Testing Methods: Breath Collection Device, BREATHID, EXALENZ

The BREATHID device is a molecular correlation spectrometer, developed by EXALENZ BIOSCIENCE LTD. EXALENZ BREATHID is FDA cleared with use of a substrate (¹³C-Urea) for the diagnosis of H. Pylori infection. This device is based on specific optical-radiation emission and absorption by ¹³CO₂ and ¹²CO₂ gases. The BREATHID continuously senses exhaled breath in real-time through a nasal cannula worn by the patient and measures ¹³CO₂ and ¹²CO₂ concentrations to establish the ¹³CO₂/¹²CO₂ ratio.

The BREATHID was used according to the instructions in the approved package labeling. BREATHID is manufactured by EXALENZ BIOSCIENCE under 510K K011668. EXALENZ FDA IDE # G080107, IDE # G110157, Pre-submission Q120223 and 510(k)# K011668—all related to the EXALENZ BREATHID device.

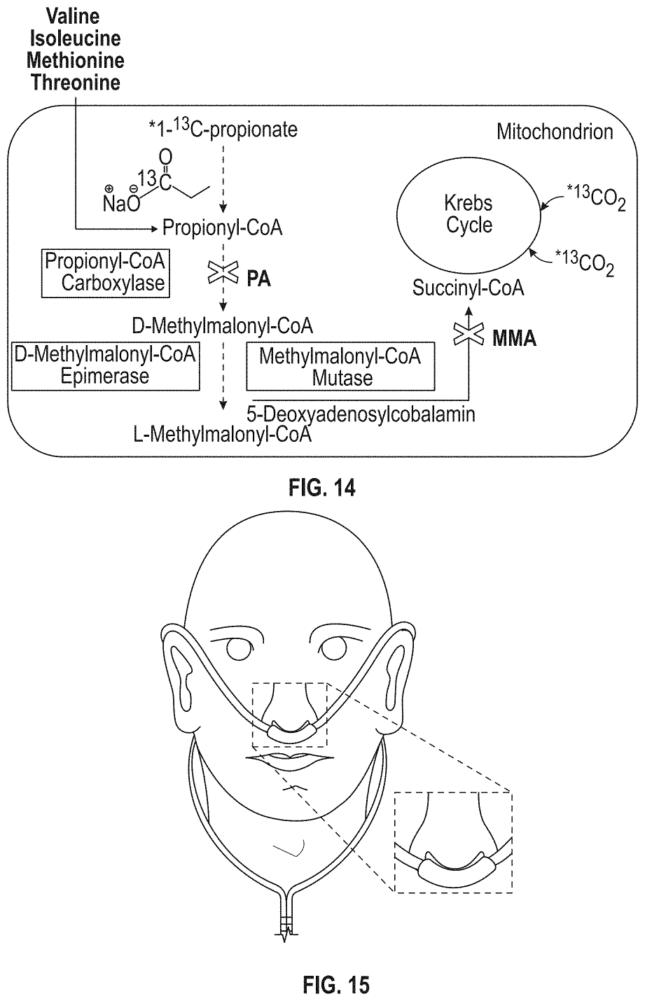

1-¹³C-labeled propionate was used to assess in vivo enzymatic activity of the propionate oxidation pathway in patients with methylmalonyl-CoA mutase or propionyl-CoA carboxylase enzymatic deficiencies, causing methylmalonic and propionic acidemia, respectively (see FIG. 14).

Comparative Example 6(a)—EASYSAMPLER Breath Test Kit, QUINTRON: A single bolus of sodium 1-¹³C-propionate was administered by mouth or via a gastrostomy tube over no more than 2 min followed by similar amount of water consumption. All participants were fasting for 3 hours prior and throughout the P^{t} hour of the procedure but were allowed access to water for PO or g-tube fluid intake, if desired. Food was offered after the first hour of breath sampling if clinically indicated. (1) The dose administered was 0.5 mg/kg dissolved in sterile water at a concentration of 1 mg/ml (99 atom % ¹³C, clinical grade, MW: 97.05 g/mol; from CAMBRIDGE ISOTOPE LABORATORIES Andover, Mass., prepared on the day of the study by the NIH pharmacy). (2) Serial breath samples were collected manually by the study personnel using disposable breath collection kits (EASYSAMPLER Breath Test Kit, QUINTRON) into vacutainer tubes at timed intervals: before isotope administration and at structured time points over 2 hours (baseline, 1, 5, 7, 10, 15, 20, 25, 30, 45, 60, 90, and 120 minutes), for measurement of ¹³CO₂. (3) The isotope ratio (¹³C/¹²C) of the expired gas was measured by a gas isotope ratio mass spectrometer (METABOLIC SOLUTIONS INC., Nashua, N.H.). Results were reported as delta: δ=(¹³C:¹²Csample/¹³C:¹²Cstandard−1)*1000. APE: Atomic percent excess: the level of isotopic abundance above a given background reading, which is considered zero. Percent Dose Oxidized at each time point=CO₂ production rate×Σ(APE (t)/(mmol C¹³ administered)×100, where CO₂ production rate was the one measured by the indirect calorimetry on the same day just prior to the isotope study.

The 1-¹³C—propionate, derived from the ingested 1-¹³C-sodium propionate, is absorbed into the blood and then exhaled in the breath. Absorption and distribution of ¹³CO₂ is fast. Therefore, the cleavage of propionate that produces the CO₂ occurs immediately after the solution is ingested and enables instant detection of increased ¹³CO₂ in the exhaled breath. The majority of the label is oxidized within 15-30 min in normal controls. In the case of severe mut° MMA or PA patients with very little to no enzyme activity, the 1-¹³C-propionate does not produce ¹³CO₂ in the liver resulting in a minimal increase over baseline in the ¹³CO₂/¹²CO₂ ratio, that often peaks 30 min to one hour after ingestion of the label.

### Example 6(b) BREATHID

The test was begun with the selection of the PATIENT MODE and with the collection of a baseline breath. The patient breathed normally while the BREATHID device collected samples through the IDcircuit™ nasal cannula. The IDcircuit™ extracted moisture and patient secretions from the breath samples to provide accurate CO₂ readings, and the device measured the ¹³CO₂/¹²CO₂ ratio of the baseline measurement.

The patient then ingested a test drink consisting of 0.5 mg/kg 1-¹³C-sodium propionate. While the patient continued to breathe normally, the BreathID® device continually and non-invasively sampled the patient's breath (via the cannula) and measured the changes in the ¹³CO₂/¹²CO₂ ratio versus the original baseline sample. These changes were displayed as a graph on the large display screen in real time while the test continued. The graph showed multiple points that allowed the physician to identify the change in the Delta Over Baseline (DOB) of the ¹³CO₂/¹²CO₂ ratio in response to the administered 1-¹³C-sodium propionate. Once the BreathID® device has collected enough data to complete the scheduled testing time (up to 2 hours), it automatically ended the test and printed out the results.

Detailed standard operating procedure (SOP): (1) It was ensured that the BreathID® device was activated on PATIENT MODE. The device mode appears on the top corner of the screen. (2) The instructions on the screen were followed. (3)(a) The IDcircuit™ was taken out of its bag and the tubing sleeve was slid down as far as it would go. The cannula tips were gently placed into the patient's nostrils, and the cannula tubing was placed over the ears, as shown in FIG. 15. (3)(b) The tubing sleeve was slid up towards the neck to fit comfortably under the chin. (3)(c) The IDcircuit™ was connected to the BreathID® device by twisting the orange connector at the free end of the cannula clockwise until it was secured into the dedicated socket of the BreathID® device. (3)(d). It was verified that the IDcircuit™ was not twisted or kinked and that the cannula tips were in the nostrils. It was ensured that the IDcircuit™ cannula tips moldings were positioned inwards. (3)(e) The OK button was clicked to proceed. The baseline values were measured by the BreathID® device and the results were shown on the screen. (4) Test drink preparation: Note: the test drink should be administered within two hours of preparation, as this is the maximal time for maintaining solution stability. The 1-¹³C-sodium propionate (0.5 mg/kg) was dissolved in lmg/ml concentration (0.5 ml/kg) of tap water in a drinking cup or oral syringe. (5) Administration of the test drink and start of measurement: Note: The drink was not administered until prompted by the screen instructions on the device (this made certain that the baseline sample had been collected properly). 5(a) It was ensured that the patient drank the solution through the straw. 5(b) The patient drank the solution within two minutes and consumed the entire amount. 5(c) After the patient finished drinking the solution, the OK button was pressed to proceed. (6) Measurement: The BreathID® device continually analyzed the trend of measured results. When the BreathID® device determined that the final value would be positive or negative, i.e. greater or less than 5 Delta Over Baseline, it automatically ended the test and printed out the results. (7) Removal and discard of the IDcircuit™: When the measurement was complete, the IDcircuit™ was disconnected from both the patient and the device. The IDcircuit™ and all other used components of the kit were disposed, according to standard operating procedures or local regulations for the disposal of used medical waste. (8) Printing Results: 8(a) After the measurement was complete, the device automatically printed the test results. The printout contained the graph as seen on the screen, including the date, time, test number and Delta Over Baseline value of the last point measured. 8(b) The printed results were torn off and patient data was filled in.

Test Results:

The ratio of CO to CO in breath samples was determined by MOLECULAR CORRELATION SPECTROMETRY (MCS™), which was utilized by the BreathID® device software. The results of the BreathID® test were provided as Delta Over Baseline. Delta Over Baseline is the difference between the Delta value (based on a ratio of ¹³CO₂/¹²CO₂) in the test specimen and the corresponding baseline sample. There were no calculations required by the user.

Validation:

Tests were conducted to evaluate the reproducibility of results obtained by the two different breath collection devices and isotope ratio measurements.

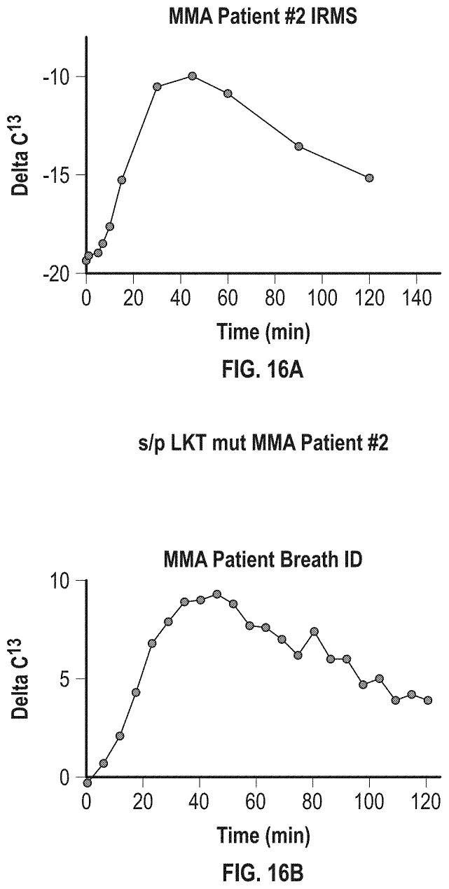

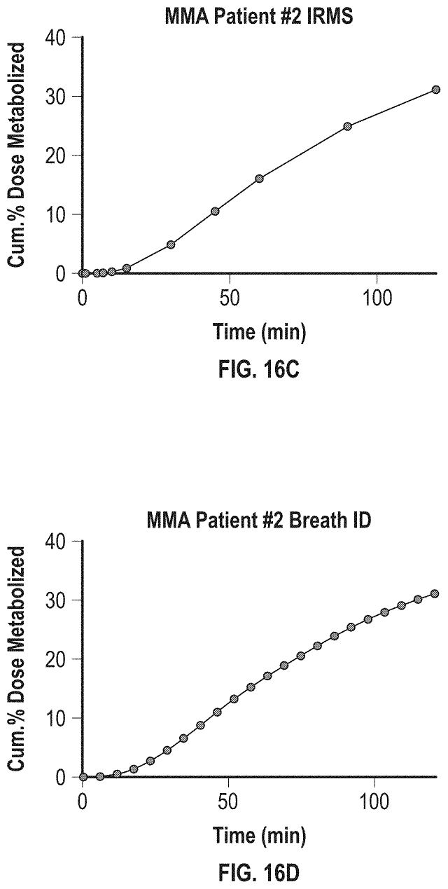

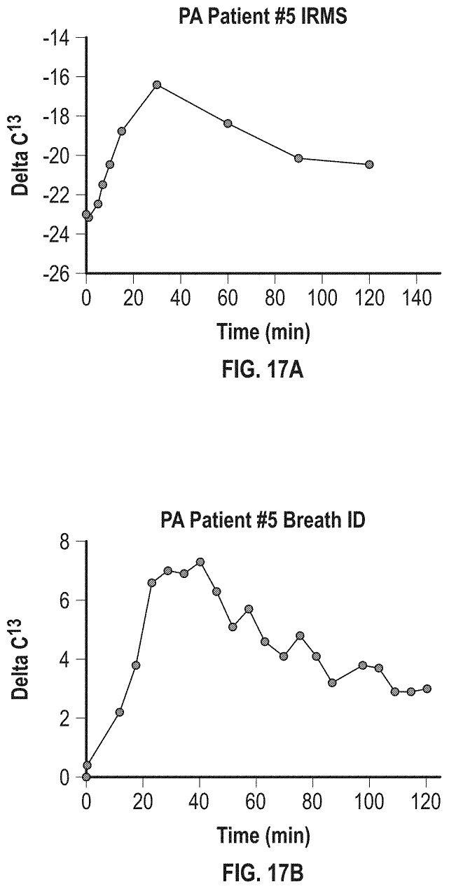

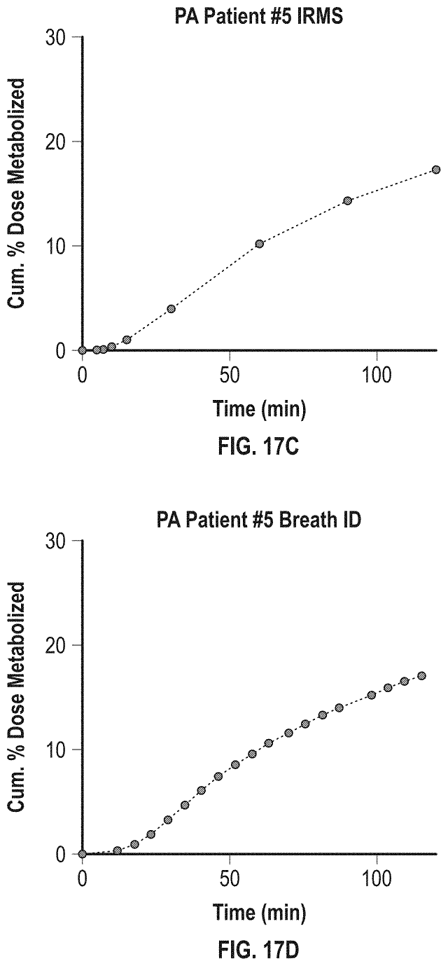

The ratio measurements of ¹³CO₂/¹²CO₂ obtained with the BreathID® device were conducted simultaneously with those obtained by the gold-standard method of Isotope Ratio Mass Spectroscopy, at METABOLIC SOLUTIONS, INC., Nashua, United States, in the same patient, with nearly identical results. Representative results demonstrating the overall high agreement from 10 patients with MMA and 8 with PA tested to date are presented at FIGS. 16A-17D.

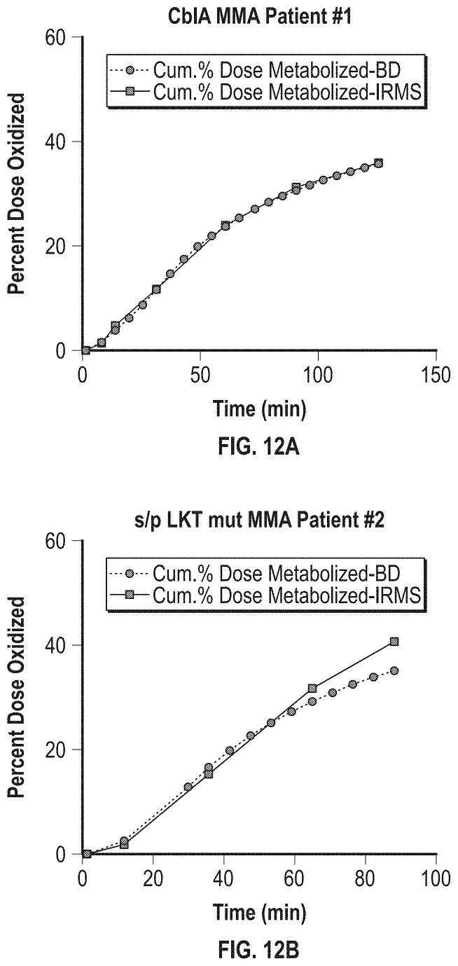

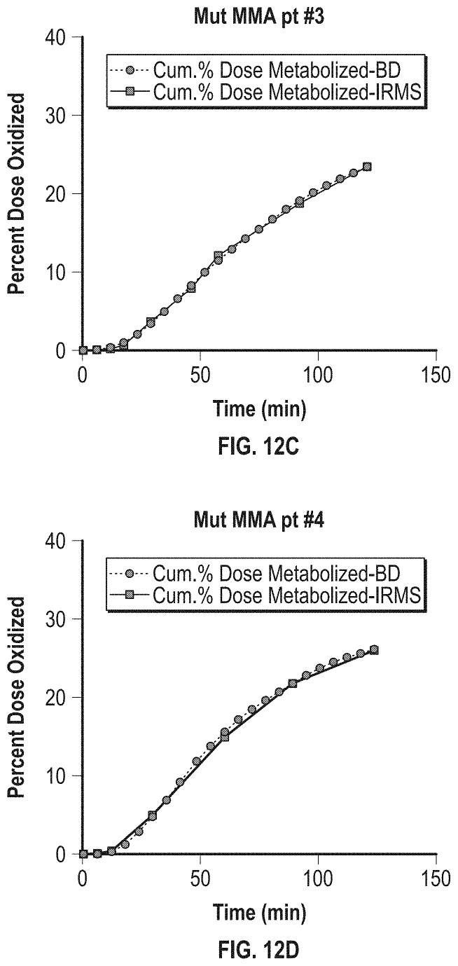

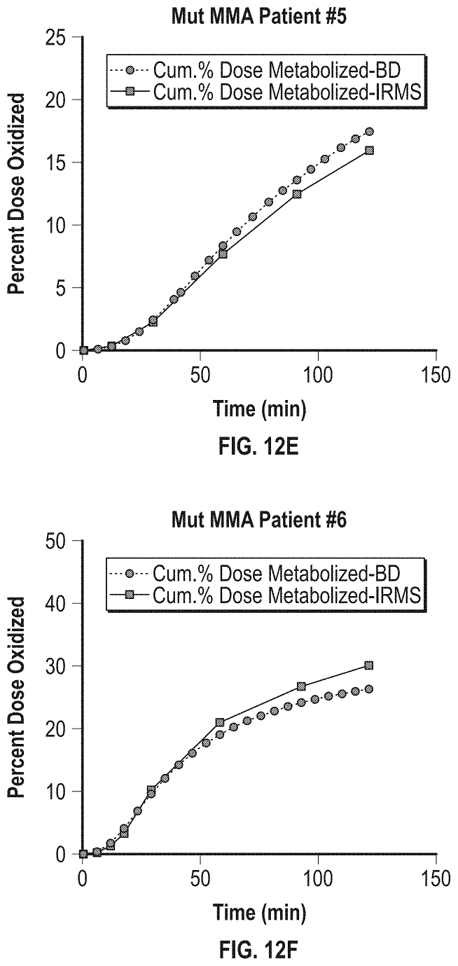

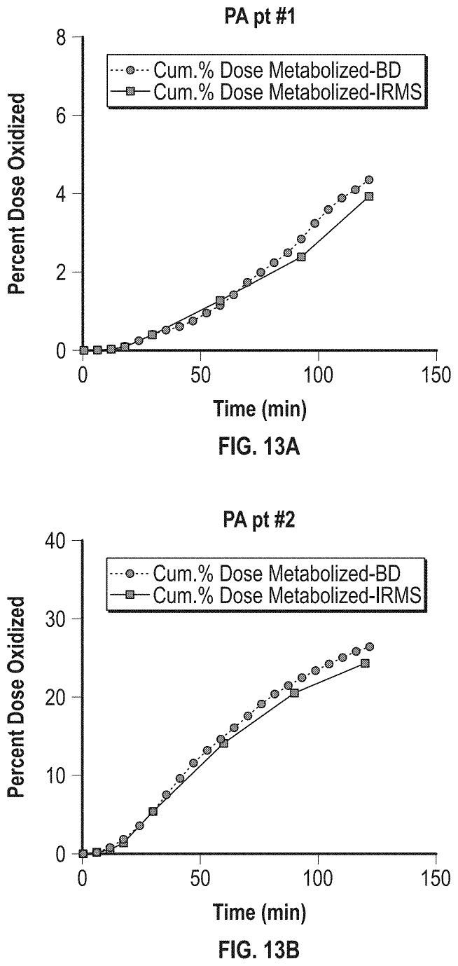

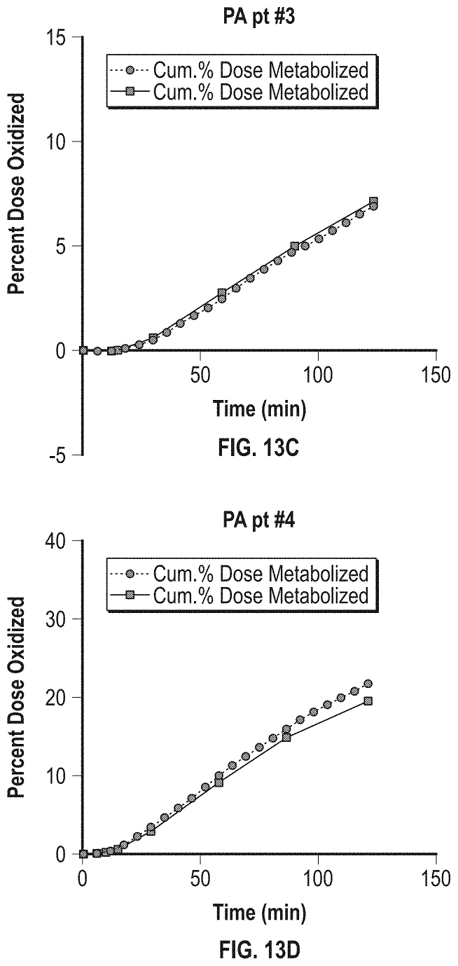

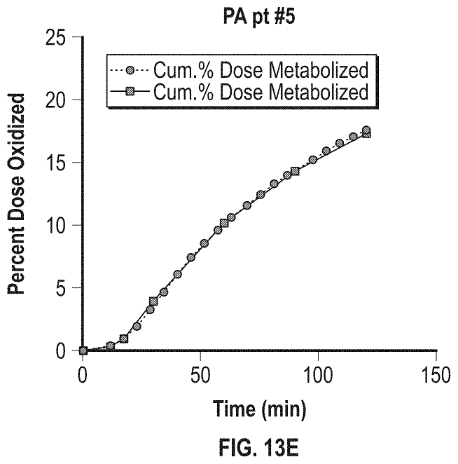

Further FIGS. 12A-13E similarly show the consistency in measurement of metabolic oxidation in MMA and PA patients with Isotope Ratio Mass Spectroscopy and BreathID®. While BreathID® provides the practical benefits of real-time monitoring not found with Isotope Ratio Mass Spectroscopy, which requires bag collection of exhalation and shipping of breath samples to a laboratory for analysis.

1. A method for determining the efficacy of a treatment for an organic acidemia in a subject, the method comprising:
prior to the treatment:
(i) administering to the subject a composition having isotope-labeled propionate;
(ii) collecting breath samples from the subject at a plurality of time points after step (i);
(iii) measuring the ¹³CO₂/¹²CO₂ ratio of the breath samples from step (ii);
(iv) determining a first isotope-labeled propionate oxidation rate based on the measured ¹³CO₂/¹²CO₂ ratio of step (iii) and measured CO₂ production rate;
following the treatment:
(v) administering to the subject a composition having isotope-labeled propionate;
(vi) collecting breath samples from the subject at a plurality of time points after step (v);
(vii) measuring the ¹³CO₂/¹²CO₂ ratio of the breath samples from step (vi);
(viii) determining a second isotope-labeled propionate oxidation rate based on the measured ¹³CO₂/¹²CO₂ ratio of step (vii) and measured CO₂ production rate; and
comparing the first isotope-labeled propionate oxidation rate with the second isotope-labeled propionate oxidation rate, wherein an increase in the second isotope-labeled propionate oxidation rate compared to the first isotope-labeled propionate oxidation rate indicates efficacy of the treatment.

2. The method of claim 1, wherein the treatment is a liver-directed treatment.

3. The method of claim 1, wherein the treatment comprises administering to the subject a liver-directed gene transfer vector.

4. The method of claim 1, wherein the treatment is liver transplantation or combined liver and kidney transplantation.

5. The method of claim 1, wherein the treatment is selected from the group consisting of gene therapy, cell therapy, small molecules, enzyme specific chaperonins, engineered microbes/microbiome, mRNA therapy, enzyme replacement therapy, genome editing, read-through agents, stem cell therapies, chaperones, ERT, or any other processes that could improve MUT or PCC activity or propionate oxidation or associated mitochondrial dysfunction.

6. The method of claim 1, wherein the organic acidemia is selected from the group consisting of methylmalonic acidemia (MMA), propionic acidemia (PA), isovaleric acidemia, glutaric aciduria type 1 (GA1), beta-ketothiolase deficiency (BKT), 3-methylcrotonyl-CoA carboxylase deficiency (3-MCC), 3-hydroxy-3-methylglutaryl-CoA lyase deficiency (HMG), 3-Methylglutaconic acidemia or 3-Methylglutaconyl-CoA Hydratase Deficiency (MGA), D-2 Hydroxyglutaric Aciduria (D2-HGA), Isobutyryl-CoA Dehydrogenase Deficiency 3-Hydroxyisobutyric aciduria (ICBD), L-2-Hydroxy-glutaricaciduria (L2HGA), Malonyl-CoA Decarboxylase Deficiency aka Maionic Acidemia (MA), Multiple carboxylase deficiency (MCD, holocarboxylase synthetase), and 3-Hydroxyisobutyryl-CoA Hydrolase Deficiency (HIBCH).

7. The method of claim 1, wherein the organic acidemia is methylmalonic acidemia or propionic acidemia.

8. The method of claim 1, wherein the organic acidemia is a disorder of propionate metabolism or a cobalamin metabolic and transport disorder causing MUT deficiency.

9. The method of claim 8, wherein the disorder of propionate metabolism is caused by isolated methylmalonyl-CoA mutase due to (MUT), MMAA, MMAB, MMADHC deficiency, or mut, cblA, cblB, cblD variant 2 classes of MMA.

10. The method of claim 8, wherein the cobalamin metabolic and transport disorders is selected from the group consisting of patients with MMACHC, MMADHC, LMBRD1, ABCD4, TC2, CD320, AMN deficiency, cblC, cblD, cblF, cblJ, TCBLR and Imerslund-Graesbeck forms of combined MMAemia-hyperhomocysteinemia.

11. The method of claim 1, wherein the organic acidemia is a disorder of propionate metabolism causing PCC deficiency.

12. The method of claim 8, wherein the disorder of propionate metabolism is caused by propionyl-CoA carboxylase deficiency (PCC) due to mutations in PCCA or PCCB.

13. The method of claim 1, wherein isotope-labeled propionate is administered in the amount of less than or equal to about 10 μmol/kg body weight.

14-16. (canceled)

17. The method of claim 1, wherein the isotope-labeled propionate is sodium 1-¹³C-propionate.

18. The method of claim 1, wherein the CO₂ production rate in step (iv) is measured by an indirect calorimetry cart on the same day prior to step (i), wherein the CO₂ production rate in step (viii) is measured by an indirect calorimetry cart on the same day prior to step.

19. The method of claim 1, wherein the composition having isotope-labeled propionate is orally administered.

20. The method of claim 1, wherein the composition having isotope-labeled propionate is administered via gastric route.

21-22. (canceled)

23. A method for improving hepatic enzyme activity in a subject having an organic acidemia, the method comprising:
prior to a treatment:
(i) administering to the subject a composition having sodium isotope-labeled propionate;
(ii) collecting breath samples from the subject at a plurality of time points after step (i);
(iii) measuring ¹³CO₂/¹²CO₂ ratio of the breath samples from step (ii);
(iv) determining a first isotope-labeled propionate oxidation rate based on the measured ¹³CO₂/¹²CO₂ ratio of step (iii) and measured CO₂ production;
administering a treatment to the subject to improve compromised hepatic enzyme activity associated with the organic acidemia after step (ii);
following the treatment:
(v) orally administering to the subject a composition having isotope-labeled propionate;
(vi) collecting breath samples from the subject at a plurality of time points after step (v);
(vii) measuring ¹³CO₂/¹²CO₂ ratio of the breath samples from step (vi);
(viii) determining a second isotope-labeled propionate oxidation rate based on the measured ¹³CO₂/¹²CO₂ ratio of step (vii) and measured CO₂ production rate;
discontinuing, altering, or continuing the treatment based on the second isotope-labeled propionate oxidation rate after treatment compared to the first isotope-labeled propionate oxidation rate before the treatment.

24. The method of claim 23, where in the enzyme is selected from the group consisting of methylmalonyl-CoA mutase, propionyl CoA carboxylase, isovaleryl-CoA dehydrogenase, Glutaryl CoA Dehydrogenase, beta-ketothiolase, 3-methylcrotonyl-CoA carboxylase, 3-hydroxy-3-methylglutaryl-CoA lyase, 3-Methylglutaconyl-CoA Hydratase, Isobutyryl-CoA Dehydrogenase, Malonyl-CoA Decarboxylase, Multiple carboxylase, and 3-Hydroxyisobutyryl-CoA Hydrolase.

25-30. (canceled)

31. A method for determining the efficacy of a treatment for an organic acidemia in a subject, the method comprising:
After the treatment:
(i) administering an isotope-labeled metabolite to the subject wherein the isotope-labeled metabolite is 1-¹³C-propionate, 1-¹³C-glycine, or 1-¹³C-methionine;
(ii) measuring a level of an isotope-labeled product of the isotope-labeled metabolite in exhaled breath of the subject following administration of the isotope-labeled metabolite;
(iii) comparing the measured level of isotope-labeled product of the isotope-labeled metabolite in the subject to a predetermined level;
wherein an increase in the measured level of isotope-labeled product compared to the predetermined level indicates efficacy of the treatment.

32-37. (canceled)
